(19) United States
(12) Patent Application Publication
(10) Pub. No.: US 2002/0099285 A1
(43) Pub. Date: Jul. 25, 2002

(54) Three-dimensional CAD system

(76) Inventor: Shinya Kano, Osaka (JP)

Correspondence Address: VARNDELL & VARNDELL, PLLC, 106-A S. COLUMBUS ST., ALEXANDRIA, VA, 22314, US

(21) Appl. No.: 10/045,086

(22) Filed: Jan. 15, 2002

(30) Foreign Application Priority Data
Jan. 19, 2001 (JP) 012349/2001

(51) Int. Cl. A61B 5/05

(52) U.S. Cl. 600/407

(57) ABSTRACT

It is enabled to integrate three-dimensional models, which are concurrently worked and created by Design Department and Production Department, to reduce a lead time. After the three-dimensional model created by Design Department is copied, a production shape addition flag is given to identification IDs (Nos. 16, 17) of parts to be added by Production Department to the copied three-dimensional model. Therefore, when the three-dimensional models of Design Department and Production Department are compared to find that they have the same identification IDs (Nos. 16, 17), it can be judged that they are parts added by Design Department and Production Department if a production shape addition flag were given to the identification IDs. And, according to the judged result, the identification IDs can be changed so that the identification IDs become different (the identification ID Nos. 16, 17 of the parts added by Production Department are changed to Nos. 19, 20 so to become different from the identification ID Nos. 16, 17, 18 of the parts added by Design Department).

### BACKGROUND OF THE INVENTION

1. Field of the Invention

The present invention relates to a three-dimensional CAD (Computer Aided Design) system.

2. Description of the Related Art

Construction machine manufacturers are trying to reduce a lead time by making a concurrent design.

Here, the concurrent design means a designing method by which addition or modification of parts with respect to the same model is concurrently performed by Design Department and Production Department.

Design Department changes a design in view of quality assurance of products. Production Department changes a design in view of product producing technologies. In other words, the three-dimensional model to be created by Design Department is a product shape itself but a mold shape for producing is different from it. Therefore, Production Department makes a correction or an addition, which is different from the one made by Design Department, of the three-dimensional model.

There is a three-dimensional CAD system which has a parametric function.

The parametric function means a function of configuring a three-dimensional model by allotting an identification ID to respective components of a three-dimensional model and overlaying a part by referencing to already overlaid parts by another part. For example, in three-dimensional model 50C shown in FIG. 25, a command (hole, through) or a parameter (identification ID 6, d, r) for “drilling a through hole having diameter 2 r at a position distance d away from the “hole” identified by shape number (identification ID) 6” is associated with a part “hole” identified by shape number (identification ID) 7. In other words, the part “hole” identified by the shape number (identification ID) 7 is related to the part “hole” which is identified by the shape number (identification ID) 6 so to reference to it.

The three-dimensional CAD system having the parametric function executes commands, which are associated with the identification IDs, in order of the identification ID numbers to reproduce the shape of the three-dimensional model. Therefore, if there are parts having the same identification ID, it becomes impossible to reproduce the three-dimensional model.

The above three-dimensional CAD system is recommended to make an operation to add or correct a part with respect to a single three-dimensional model by Design Department and Production Department together. It is to avoid overlap of parts having the same identification ID as described above.

Referring to FIGS. 31(a) to 31(c), which are parts of FIG. 31, disadvantages of prior art will be described. FIGS. 31(a) to 31(c) exemplify an operation of adding parts to the original three-dimensional model which is comprised of parts having identification ID Nos. 1 to 15.

The original three-dimensional model created by Design Department is copied by Production Department.

Design Department adds parts (design shapes) having identification ID Nos. 16 to 18 to the original three-dimensional model to create a new three-dimensional model.

Meanwhile, Production Department adds parts (production shape) having identification ID Nos. 16, 17 to the copied three-dimensional model to create a new three-dimensional model.

Lastly, in order to create a three-dimensional model incorporating the parts added by both Departments, the three-dimensional model finally created by Design Department is copied by Production Department, and the respective parts having the identification ID Nos. 16, 17 added by Production Department are consolidated (integrated) to the copied three-dimensional model, but it is impossible to consolidate into a single three-dimensional model because the identification ID Nos. 16, 17 overlap. In other words, the three-dimensional models created by both Departments cannot be brought together in one file.

FIGS. 32(a) to 32(c), which are parts of FIG. 32, show an example of an operation to make addition or correction of the parts with respect to the original three-dimensional model comprised of the respective parts having the identification ID Nos. 1, 2, 3 (shape 1, shape 2, shape 3).

The original three-dimensional model created by Design Department is copied by Production Department.

Design Department adds the respective parts (design shapes) having the identification ID Nos. 4, 5 to the original three-dimensional model and also corrects the part having the identification ID No. 2 to create a new three-dimensional model.

Meanwhile, Production Department adds the respective parts (production shapes) having the identification ID Nos. 4, 5 to the copied three-dimensional model to create a new three-dimensional model.

Lastly, in order to integrate the three-dimensional models, the contents of the parts configuring the three-dimensional models created by both Departments are compared for the parts which are given the same identification ID.

As to the identification ID 2 (shape 2), “the parts have different contents and the same identification ID between both Departments”, so that it is possible to identify correctly, “the parts were corrected”.

But, as to the identification ID 4 (shape 4), even if different parts are added by both Departments, it is identified erroneously, “the parts were corrected” because “the parts have different contents and the same identification ID between both Departments” in the same way as the parts are corrected. In other words, the prior art cannot identify properly whether the parts are added or corrected when the three-dimensional models are integrated, so that it is impossible to integrate the three-dimensional models.

Thus, even if Design Department and Production Department proceed the operation at the same time, it is impossible to integrate the three-dimensional models which are finally created by both Departments. Therefore, the file of the original three-dimensional model is locked so that it is not copied to proceed the operations by Design Department and Production Department at the same time.

Meanwhile, Design Department and Production Department are actually difficult to cooperate each other to proceed designing of a single three-dimensional model.

Therefore, it is necessary to have an operation of adding or correcting the parts with respect to the three-dimensional model to which a part was added or corrected by Design Department, and it is impossible to have the simultaneous operation form. As a result, a lead time can not be reduced.

In view of the circumstances described above, it is an object of the present invention to reduce a lead time by making it possible to integrate three-dimensional models which are created by a simultaneous operation by Design Department and Production Department.

### SUMMARY OF THE INVENTION

A first aspect of the invention is directed to a three-dimensional CAD system which allots identification IDs to respective component parts, references to already overlaid parts to overlay parts so to configure an original three-dimensional model, copies the original three-dimensional model, and concurrently performs addition of parts to the original three-dimensional model and addition of parts to the copied three-dimensional model, to integrate the original three-dimensional model and the copied three-dimensional model into the same three-dimensional model, wherein:

a flag is added to identification IDs of the parts to be added to the copied three-dimensional model after copying the original three-dimensional model; and

when the processing of adding the parts to the original three-dimensional model and the processing of adding the parts to the copied three-dimensional model are performed and there is a difference in the contents of the parts having the same identification ID, the identification ID is changed according to the flag so that the identification ID of the part added to the original three-dimensional model and the identification ID of the part added to the copied three-dimensional model become different, and the original three-dimensional model and the copied three-dimensional model are integrated into the same three-dimensional model.

According to the first aspect of the invention, three-dimensional model 30 created by Design Department is copied and a production shape addition flag is given to identification IDs (Nos. 16, 17) of parts to be added by Production Department to the copied three-dimensional model 30A as shown in FIGS. 10(a) to 10(d). Therefore, the three-dimensional models of Design Department and Production Department are compared and, if they have the same identification IDs (Nos. 16, 17) and a production shape addition flag is given to the identification IDs, the parts can be judged as the parts added by both of Design Department and Production Department. Based on the judged result, the identification IDs can be changed so to have different identification IDs (the identification ID Nos. 16, 17 of the parts added by Production Department are changed to Nos. 19, 20 so that they become different from the identification ID Nos. 16, 17, 18 of the parts added by Design Department). As a result, the three-dimensional models which are concurrently worked and created by Design Department and Production Department can be integrated, and a lead time can be reduced considerably.

A second aspect of the invention is directed to a three-dimensional CAD system which allots identification IDs to respective component parts, references to already overlaid parts to overlay the part so to configure an original three-dimensional model, copies the original three-dimensional model, and concurrently performs addition and correction of parts to the original three-dimensional model and addition of parts to the copied three-dimensional model, to integrate the original three-dimensional model and the copied three-dimensional model into the same three-dimensional model, wherein:

a first flag is given to the identification ID of the part configuring the original three-dimensional model at a time when the original three-dimensional model is copied;

a second flag is given to the identification ID of the part to be added to the copied three-dimensional model after the original three-dimensional model is copied; and

when the processing of adding and correcting the parts to the original three-dimensional model and the processing of adding the parts to the copied three-dimensional model are performed and there is a difference in the contents of the parts having the same identification ID, the part having the identification ID which is given the first flag is judged as a part corrected in the original three-dimensional model, the part having the identification ID which is given the second flag is judged as a part added to the copied three-dimensional model, the identification ID is changed so that the identification ID of the part added to the original three-dimensional model and the identification ID of the part added to the copied three-dimensional model become different, and the original three-dimensional model and the copied three-dimensional model are integrated into the same three-dimensional model.

According to the second aspect of the invention, at a time when the three-dimensional model 30 created by Design Department is copied as shown in FIGS. 10(a) to 10(d), the design shape completion flag (first flat) is given to the identification IDs (Nos. 1 to 15) of the parts configuring the three-dimensional model 30A, the three-dimensional model 30 created by Design Department is copied, and the production shape addition flag (second flag) is given to the identification IDs (Nos. 16, 17) of the parts to be added by Production Department to the copied three-dimensional model 30A.

Therefore, the three-dimensional models 30B, 30C of Design Department and Production Department are compared, and if they have the same identification ID and the design shape addition flat (first flag) is given to its identification IDs (Nos. 1 to 15), they can be judged as parts corrected by Design Department, and if the production shape addition flag (second flag) is given to the identification IDs (Nos. 16, 17), they can be judged as parts added by Production Department. When they are judged as the parts added by Production Department, the identification IDs can be changed so to be different (the identification ID Nos. 16, 17 of the parts added by Production Department are changed to Nos. 19, 20 so that they become different from the identification ID Nos. 16, 17, 18 of the parts added by Design Department). Thus, according to the second aspect of the invention, the parts having the same identification ID can be recognized clearly whether they are corrected or added. As a result, the three-dimensional models which are concurrently worked and created by Design Department and Production Department can be integrated, and a lead time can be reduced considerably.

A third aspect of the invention is directed to a three-dimensional CAD system which allots identification IDs to respective component parts, references to already overlaid parts to overlay the part so to configure an original three-dimensional model, copies the original three-dimensional model, and concurrently performs addition and correction of parts to the original three-dimensional model by a first design department and addition and correction of parts to the three-dimensional model copied by a second design department, to integrate the original three-dimensional model of the first design department and the three-dimensional model copied by the second design department into the same three-dimensional model, wherein:

a first flag is given to the identification ID of the part configuring the original three-dimensional model at a time when the original three-dimensional model is copied for a first time;

a second flag is given to the identification ID of the part to be added to the copied three-dimensional model after the original three-dimensional model is copied for the first time;

a third flag is given to the identification ID of the part configuring the original three-dimensional model at a time when the original three-dimensional model is copied for a second time;

the identification ID of the part configuring the three-dimensional model copied for the first time and the identification ID of the part configuring the three-dimensional model copied for the second time are compared and the contents are compared for differences;

it is judged that a part is not added or corrected when the first and third flags are given but the second flag is not given and there is not a content difference;

it is judged that the part is corrected by the first design department when the first and third flags are given but the second flag is not given and there is a content difference;

it is judged that the part is added by the first design department when the third flag is given but the first and second flags are not given;

it is judged that the part is added by the second design department when the second flag is given but the first and third flags are not given;

when the second and third flags are given but the first flag is not given and there is a content difference, it is judged that the parts are added by the first and second design departments, and the identification ID is changed so that the identification ID of the part becomes different; and

the original three-dimensional model of the first design department and the three-dimensional model copied by the second design department are integrated into the same three-dimensional model.

According to the third aspect of the invention, at a time when three-dimensional model 30′ created by Design Department is copied for a first time as shown in FIGS. 15(a) to 15(c), the old design flag (first flag) is given to the identification IDs (Nos. 1 to 4) of the parts configuring the three-dimensional model 30′A, the three-dimensional model 30′ created by Design Department is copied, the production flag (second flag) is given to the identification IDs (Nos. 5, 6) of the parts to be added by Production Department to the copied three-dimensional model 30′A, and when the three-dimensional model 30′B created by Design Department is copied for a second time, a new design flag (third flag) is given to the identification IDs (Nos. 5, 6) of the parts configuring the three-dimensional model 30′B.

Therefore, identification IDs of the parts configuring the three-dimensional model 30′C copied for the first time and updated by Production Department and identification IDs of the parts configuring three-dimensional model 30′B of Design Department copied for the second time are compared, and the contents are also compared for differences. As a result, when the first flag and the third flag are given but the second flag is not given and there is no content difference (identification ID Nos. 1, 2, 4), it can be judged that no part is added or corrected. When the first flag and the third flag are given, the second flag is not given and there is a content difference, they can be judged as the parts corrected by Design Department. When the third flag is given but the first flag and the second flag are not given, they can be judged as the parts added by Design Department. When the second flag is given but the first flag and the third flag are not given, they can be judged as the parts added by Production Department. When the second flag and the third flag are given but the first flag is not given and there is a content difference (identification ID Nos. 5, 6), they can be judged as the parts added by both of Design Department and Production Department. When they are judged as the parts added by both of Design Department and Production Department, the identification IDs can be changed so to be different from the identification IDs of the parts (the identification ID Nos. 5, 6 of the parts added by Production Department are changed to Nos. 7, 8 so to be different from the identification ID Nos. 5, 6 of the parts added by Design Department). Thus, according to the third aspect of the invention, the pertinent parts can be recognized easily whether they are added by Design Department or Production Department.

As a result, it becomes possible to integrate the three-dimensional models which were concurrently worked and created by Design Department and Production Department, and a lead time can be reduced considerably.

A fourth aspect of the invention relates to the first, second or third aspect of the invention, wherein: the first part has a relationship to refer to the second part and, when only the first part of these parts is integrated; data that the identification ID of the second part is unnecessary is stored, and the identification IDs of both of the parts are changed; the identification ID of the second part is deleted according to the stored data; and the reference relationship is changed so that the first part refers to another part.

According to the fourth aspect of the invention, as shown in FIGS. 22(a) to 22(c), when hole 53 (first part) configuring three-dimensional model 50 has a relationship to refer to hole 52 (second part) (identification ID No. 7 refers to identification ID No. 6) and the hole 53 (first part) among them is integrated, data that identification ID (No. 6) of the hole 52 (second part) is unnecessary is stored, processing (to change the identification ID Nos. 6, 7 to Nos. 7, 8) to change the identification IDs of both the holes 52, 53 is performed. Then, processing to delete the identification ID (No. 7) of the hole 52 (second part) is performed according to the stored data, and processing (the identification ID No. 8 references to the identification ID No. 6) to change a reference relation is performed so that the hole 53 (first part) references to rib 51 of another part, plate, so that only some parts among the plurality of parts added by Production Department can be integrated into the three-dimensional model created by Design Department.

### BRIEF DESCRIPTION OF THE DRAWINGS

FIG. 1 is a diagram showing a display screen perspectively showing a three-dimensional model of an embodiment;

FIG. 2 is a diagram showing a display screen perspectively showing a three-dimensional model of the embodiment;

FIG. 3 is a diagram showing a display screen perspectively showing a three-dimensional model of the embodiment;

FIG. 4 is a diagram showing a display screen perspectively showing a three-dimensional model of the embodiment;

FIG. 5 is a diagram showing a display screen perspectively showing three-dimensional models of the embodiment;

FIG. 6 is a diagram showing a display screen perspectively showing three-dimensional models of the embodiment;

FIG. 7 is a diagram showing a display screen perspectively showing three-dimensional models of the embodiment;

FIG. 8 is a diagram showing a display screen perspectively showing three-dimensional models of the embodiment;

FIG. 9 is a diagram showing a display screen perspectively showing a three-dimensional model of the embodiment;

FIG. 10 is the integrated diagram of FIGS. 10(a) to 10(d);

FIGS. 10(a) to 10(d) are flow charts showing a processing procedure of a first embodiment;

FIG. 11 is the integrated diagram of FIGS. 11(a) to 11(c);

FIGS. 11(a) to 11(c) are flow charts showing a processing procedure of a third embodiment;

FIG. 12 is the integrated diagram of FIGS. 12(a) to 12(c);

FIGS. 12(a) to 12(c) are flow charts showing the processing procedure of the third embodiment;

FIG. 13 is a flow chart showing the processing procedure of the third embodiment;

FIG. 14 is the integrated diagram of FIGS. 14(a) to 14(c);

FIGS. 14(a) to 14(c) are flow charts showing a processing procedure of a second embodiment;

FIG. 15 is the integrated diagram of FIGS. 15(a) to 15(c);

FIGS. 15(a) to 15(c) are flow charts showing a processing procedure of the third embodiment;

FIG. 16 is a diagram showing a display screen perspectively showing a three-dimensional model of an embodiment;

FIG. 17 is a diagram showing a display screen perspectively showing a three-dimensional model of the embodiment;

FIG. 18 is a diagram showing a display screen perspectively showing a three-dimensional model of the embodiment;

FIGS. 19(a) and 19(b) are diagrams showing a display screen perspectively showing three-dimensional models of the embodiment;

FIGS. 20(a) and 20(b) are diagrams showing a display screen perspectively showing three-dimensional models of the embodiment;

FIG. 21 is a diagram showing a display screen perspectively showing a three-dimensional model of the embodiment;

FIG. 22 is the integrated diagram of FIGS. 22(a) to 22(c);

FIGS. 22(a) to 22(c) are flow charts showing a processing procedure of a fourth embodiment;

FIG. 23 is a diagram showing a display screen perspectively showing a three-dimensional model of the embodiment;

FIG. 24 is a diagram showing a display screen perspectively showing a three-dimensional model of the embodiment;

FIG. 25 is a diagram showing a display screen perspectively showing a three-dimensional model of the embodiment;

FIG. 26 is a diagram showing a display screen perspectively showing the three-dimensional model of the embodiment;

FIG. 27 is a diagram showing a display screen perspectively showing three-dimensional models of the embodiment;

FIG. 28 is a diagram showing a display screen perspectively showing three-dimensional models of the embodiment;

FIG. 29 is a diagram showing a display screen perspectively showing three-dimensional models of the embodiment,

FIG. 30 is a diagram showing a display screen perspectively showing three-dimensional models of the embodiment;

FIG. 31 is the integrated diagram of FIGS. 31(a) to 31(c);

FIGS. 31(a) to 31(c) are diagrams illustrating prior art technology;

FIG. 32 is the integrated diagram of FIGS. 32(a) to 32(c); and

FIGS. 32(a) to 32(c) are diagrams illustrating prior art technology.

### DETAILED DESCRIPTION OF THE PREFERRED EMBODIMENTS

Embodiments of a three-dimensional CAD system will be described with reference to the accompanying drawings.

First Embodiment:

It is assumed in this embodiment that addition of parts to a three-dimensional model is concurrently performed by Design Department and Production Department.

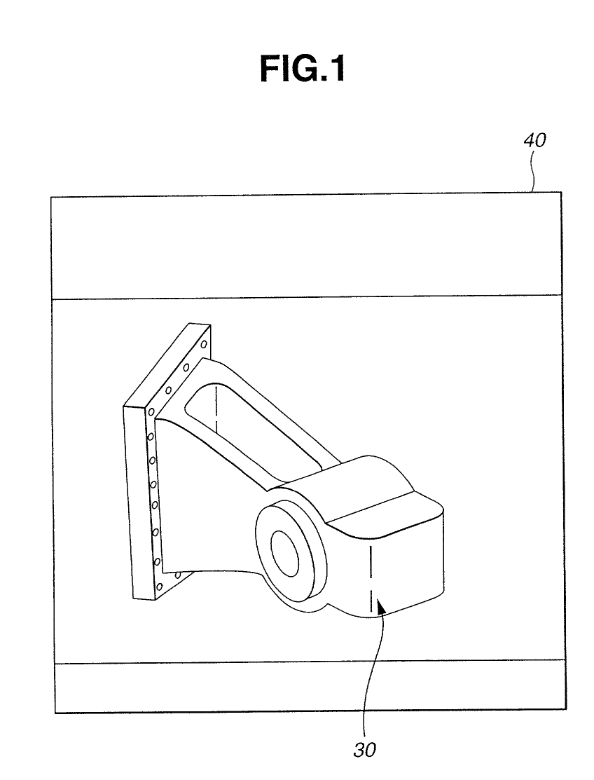

It is assumed in this embodiment that the three-dimensional model 30 of a construction machine bracket is configured by a three-dimensional CAD system as shown in FIG. 1.

FIG. 1 to FIG. 9 show contents of display screen 40 of a personal computer provided in Design Department and Production Department.

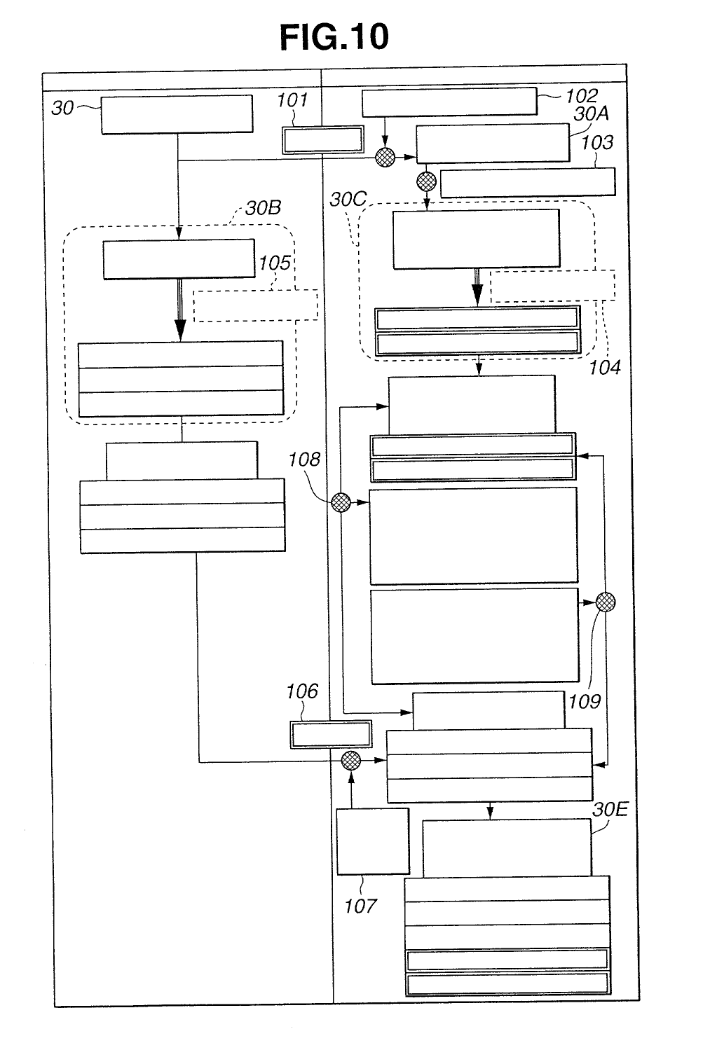

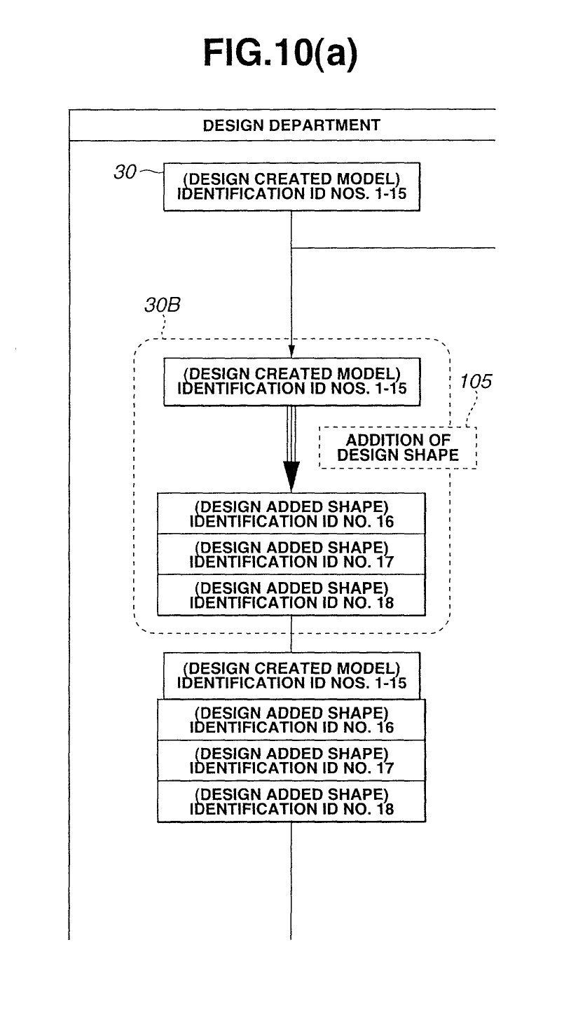

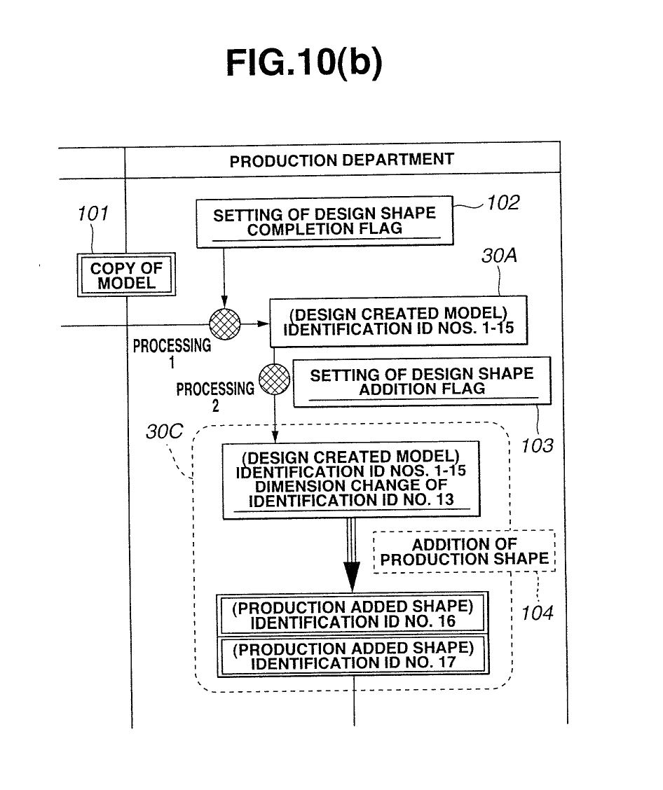

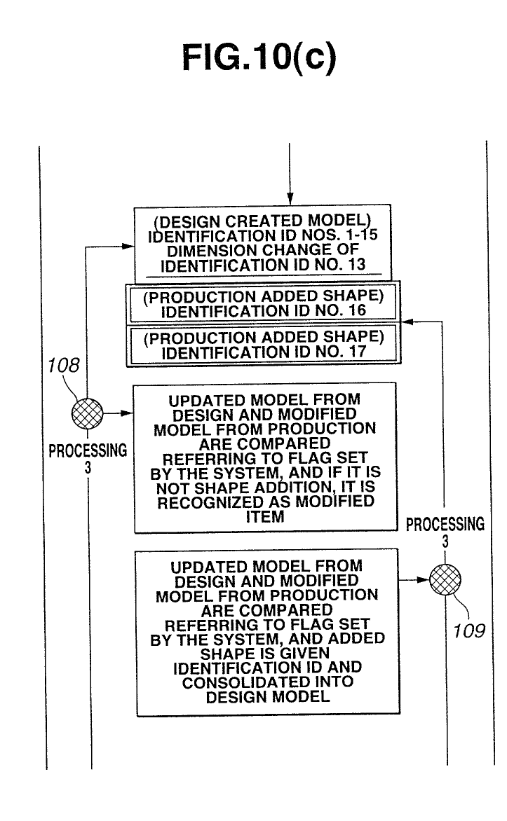

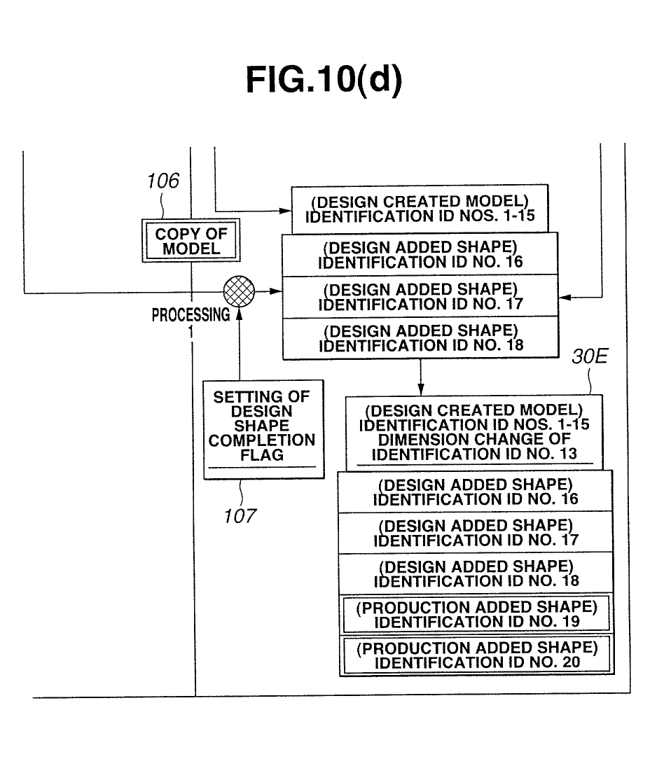

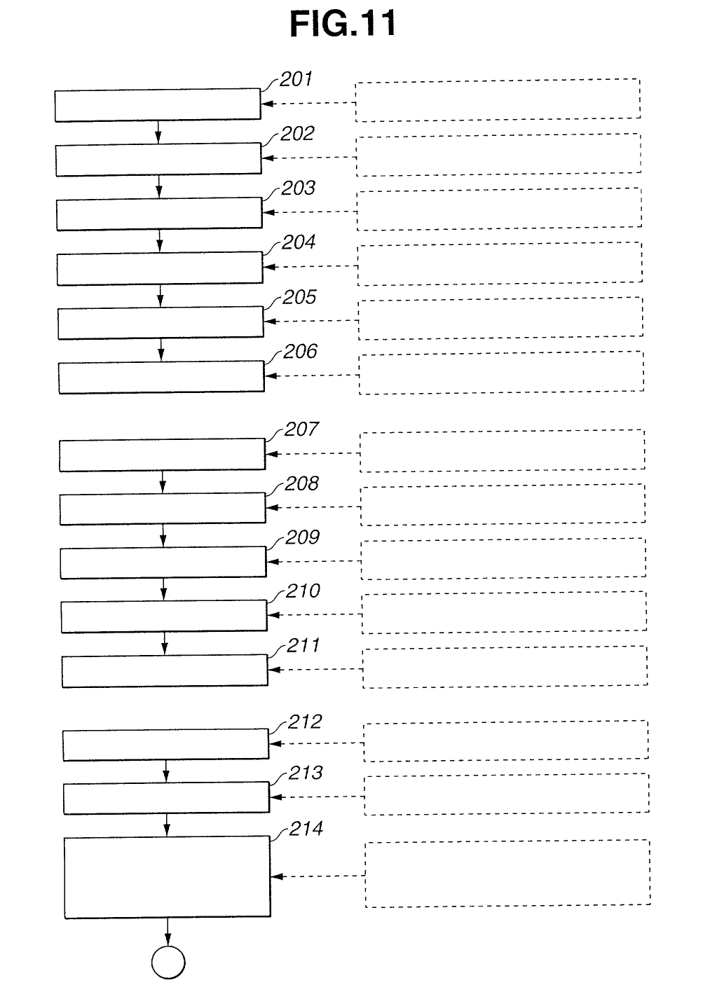

FIGS. 10(a) to 10(d), which are parts of FIG. 10, are flow charts schematically showing a processing procedure of the embodiment. FIGS. 10(a) to 10(d) show a flow of operations to integrate a three-dimensional model by adding parts to the original three-dimensional model 30 which is comprised of parts with identification ID Nos. 1 to 15.

Details will be described with reference to the drawings.

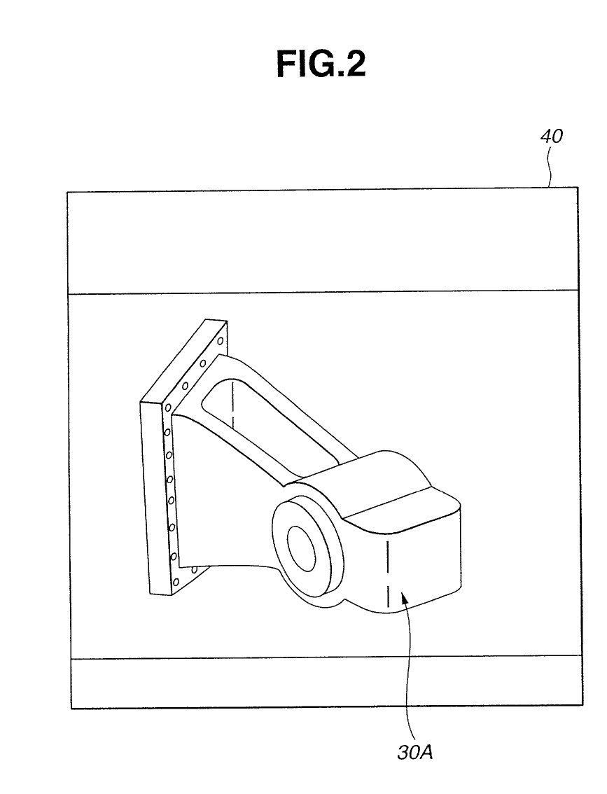

As shown in FIGS. 10(a) to 10(d), the original three-dimensional model 30 created by Design Department is copied by Production Department (step 101). FIG. 1 shows perspectively the original three-dimensional model 30 created by Design Department. FIG. 2 shows perspectively three-dimensional model 3 OA copied by Production Department. The three-dimensional models 30,30A are configured by allotting identification ID Nos. 1 to 15 to the component parts, and overlaying the parts while referencing to the already overlaid parts by other parts. According to the three-dimensional CAD system, commands associated with the identification ID Nos. 1 to 15 are executed in order of the identification ID Nos. 1, 2, 3, . . . , 15 to reproduce the shapes of the three-dimensional models 30, 30A.

Then, a design shape completion flag is set for the identification ID Nos. 1 to 15 of the parts configuring the copied three-dimensional model 30A (step 102).

Then, a production shape addition flag is set. In other words, the production shape addition flag is given to the identification IDs of the parts to be added to the three-dimensional model 30A by Production Department (step 103).

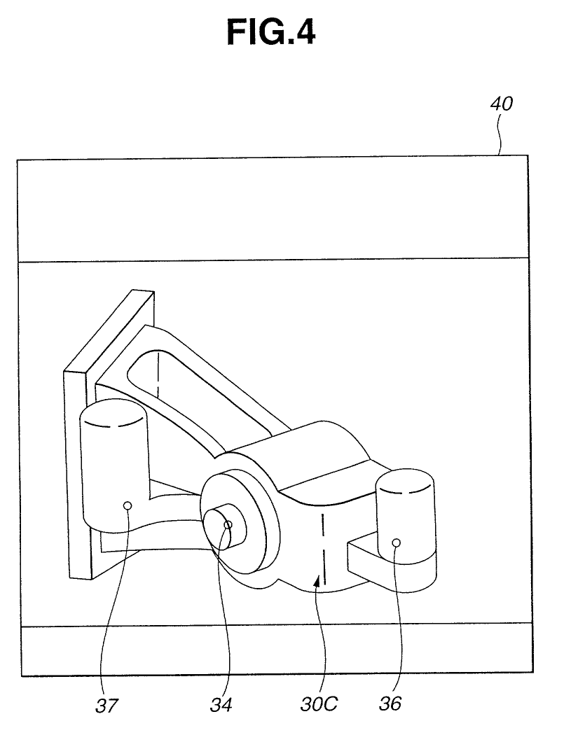

In Production Department, production shapes of the identification ID Nos. 16, 17 are newly added. As shown in FIG. 4, parts such as core 34 and deadheads 36, 37 are added to the three-dimensional model 30A of FIG. 2 to create new production three-dimensional model 30C. Identification ID No. 16 is allotted to the core 34, and identification ID No. 17 is allotted to the deadheads 36, 37. A production shape addition flag is set for the identification ID Nos. 16, 17. The part having the identification ID No. 13 is corrected to change dimensions (step 104).

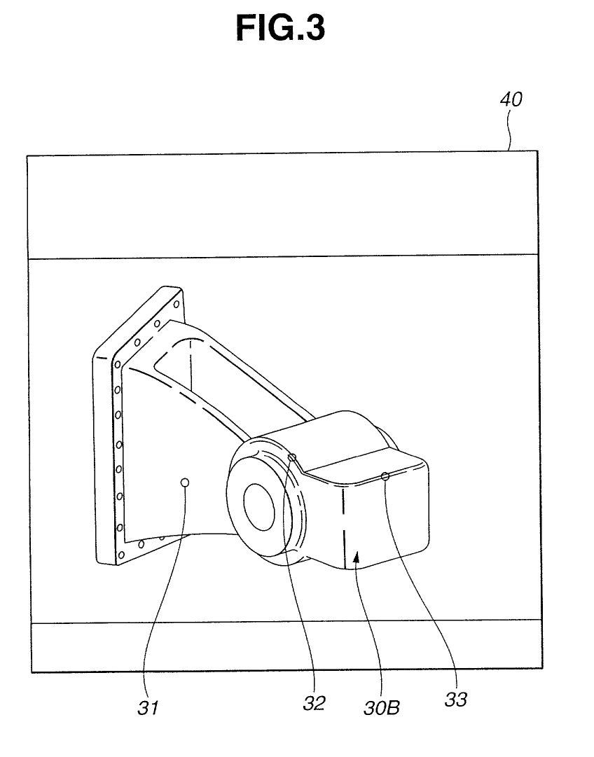

Meanwhile, Design Department adds parts (design shapes) having identification ID Nos. 16 to 18 to the original three-dimensional model 30 to create new three-dimensional model 30B. Specifically, parts called draft 31, round 32 and round 33 are added to the three-dimensional model 30 of FIG. 1 to create the new three-dimensional model 30B as shown in FIG. 3. The draft 31 is formed on one side of the main body of a bracket. The round 32 is formed on a boss. The round 33 is formed on a ridge line. Identification ID No. 16 is allotted to the draft 31, identification ID No. 17 is allotted to the round 32, and identification ID No. 18 is allotted to the round 33 (step 105).

The design updated three-dimensional model 30B to which the parts are added by Design Department is copied by Production Department (step 106).

Then, a design shape completion flag is set for the identification ID Nos. 1 to 18 of the parts configuring the copied three-dimensional model 30B in the same way as in step 102 (step 107).

Then, the design updated three-dimensional model 30B created by Design Department and the production three-dimensional model 30C created by Production Department are compared for each and same identification ID, and it is determined whether its parts are corrected or added according to the flag given to the same identification ID.

In other words, a design shape completion flag is given to the identification ID No. 13 for both the design updated three-dimensional model 30B and the production three-dimensional model 30C. The part having the identification ID No. 13 is changed its dimensions by Production Department, so that its details are different between the design updated three-dimensional model 30B and the production three-dimensional model 30C. Thus, when logic, “There are a design shape completion flag and a content difference.” is approved, the part having the identification ID No. 13 is identified as “corrected” (step 108).

Meanwhile, a design shape completion flag is given to the identification ID Nos. 16, 17 of the design updated three-dimensional model 30B, and a production shape addition flag is given to the identification ID Nos. 16, 17 of the production three-dimensional model 30C. Because the parts having the identification ID Nos. 16, 17 were separately added by Design Department and Production Department, the details of the parts are different between the design updated three-dimensional model 30B and the production three-dimensional model 30C. Thus, when logic, “there are a design shape completion flag, a production shape addition flag and a content difference” is approved, the parts having the identification ID Nos. 16, 17 are identified as “added”.

Then, processing of renumbering (changing) the identification ID is executed so to have a different identification ID between the design updated three-dimensional model 30B and the production three-dimensional model 30C. In other words, the identification ID Nos. 16, 17 of the production three-dimensional model 30C are renumbered as identification ID Nos. 19, 20 (step 109).

Therefore, both the models can be integrated by adding the contents, which were added or corrected for the production three-dimensional model 30C, to the design updated three-dimensional model 30B.

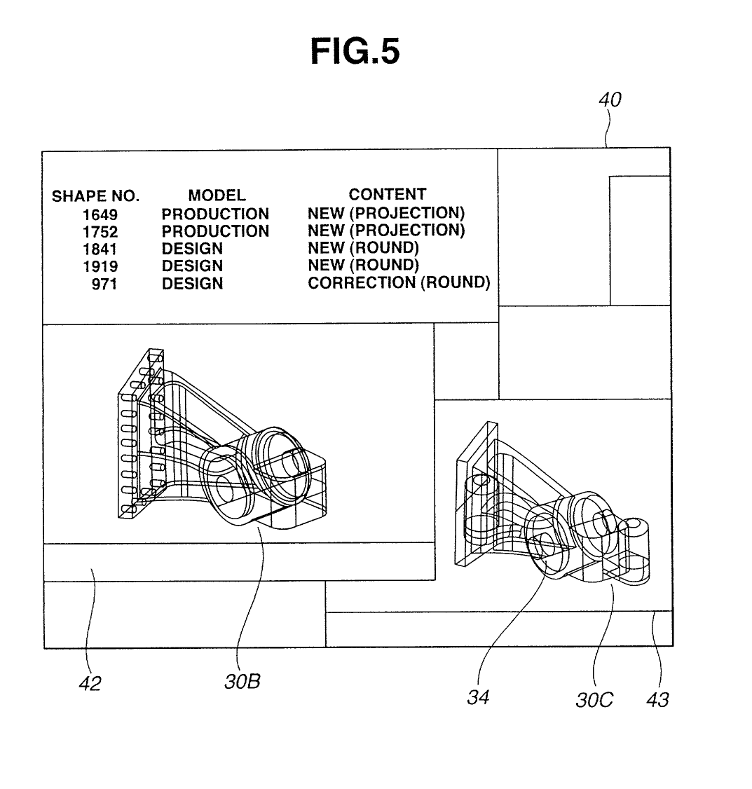

Referring to FIG. 5 to FIG. 9, processing of integrating three-dimensional models will be described briefly.

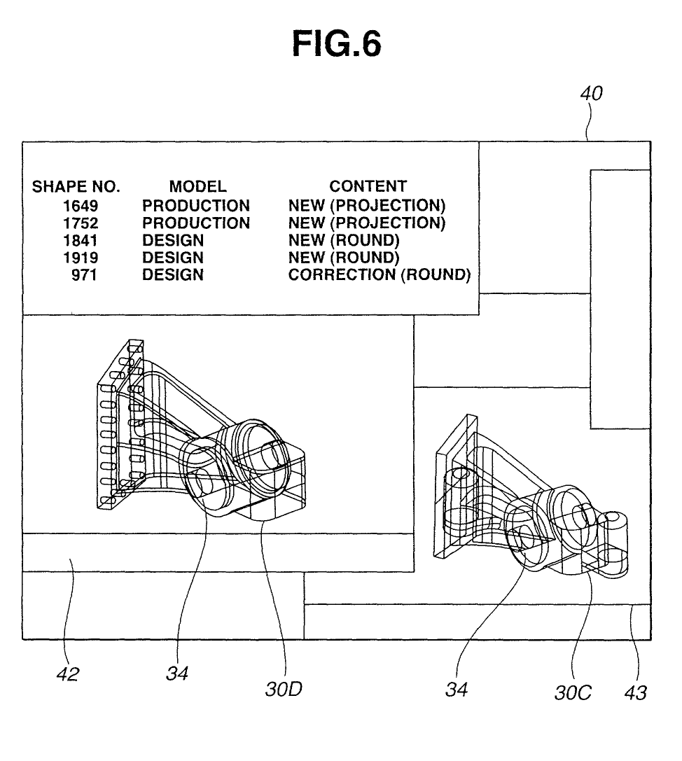

As shown in FIG. 5, window screen 42 showing the design updated three-dimensional model 30B and window screen 43 showing the production three-dimensional model 30C are arranged on the display screen 40. By comparing these window screens 42 and 43, a type of part to be consolidated can be judged with ease. Therefore, a command for consolidating the core 34, which is in the production three-dimensional model 30C shown in the window screen 43, into the design updated three-dimensional model 30B shown in the window screen 42 is input to the computer. As a result, consolidation three-dimensional model 30D into which the core 34 is consolidated is created on the window screen 42 as shown in FIG. 6.

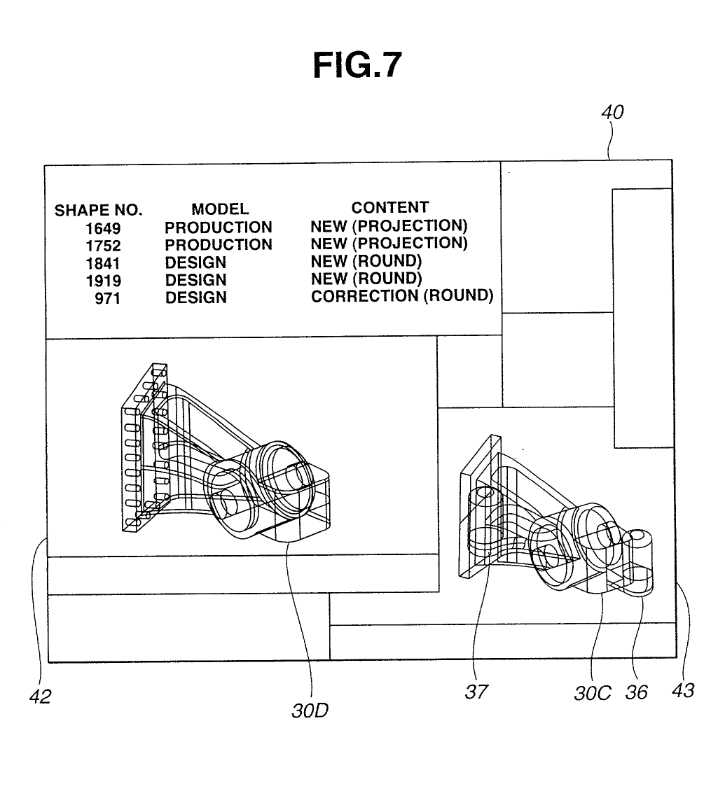

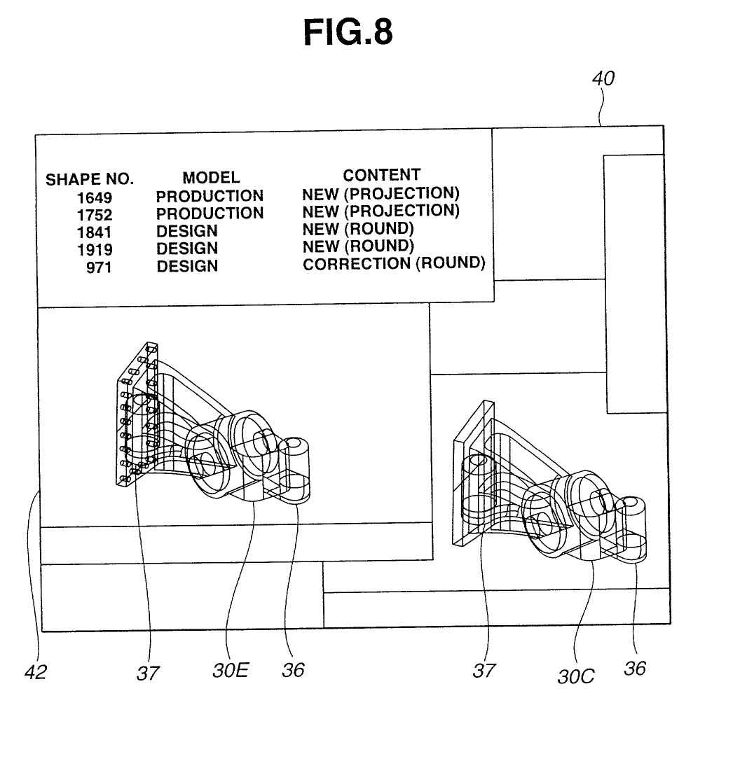

Then, the window screen 42 in which the consolidated three-dimensional model 30D is shown and the window screen 43 in which the production three-dimensional model 30C is shown are arranged on the display screen 40 as shown in FIG. 7. By comparing these window screens 42 and 43, a type of part to be consolidated next can be judged with ease. And, a command for consolidating the deadheads 36, 37 which are seen in the production three-dimensional model 30C shown in the window screen 43 into the consolidated three-dimensional model 30D shown in the window screen 42 is input to the computer. As a result, consolidated three-dimensional model 30E into which the deadheads 36, 37 are consolidated in addition to the core 34 is created on the window screen 42 as shown in FIG. 8.

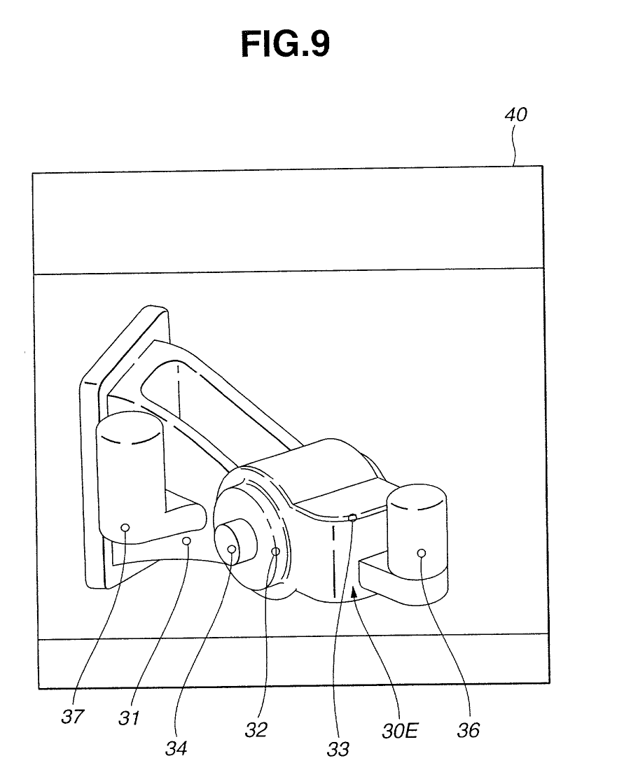

FIG. 9 shows the consolidation three-dimensional model 30E which has finally consolidated the design updated three-dimensional model 30B and the production three-dimensional model 30C.

As described above, it is configured according to the embodiment that the design shape completion flag (a first flag) is given to the identification ID of the part configuring the three-dimensional model when the three-dimensional model created by Design Department is copied, after the three-dimensional model created by Design Department is copied, the production shape addition flag (a second flag) is given to the identification ID of the part to be added by Production Department to the copied three-dimensional model. Thus, it can be recognized clearly whether the parts having the same identification ID have been corrected or added. Therefore, it becomes possible to integrate the three-dimensional models which have been processed and created by Design Department and Production Department concurrently, and a lead time can be reduced considerably.

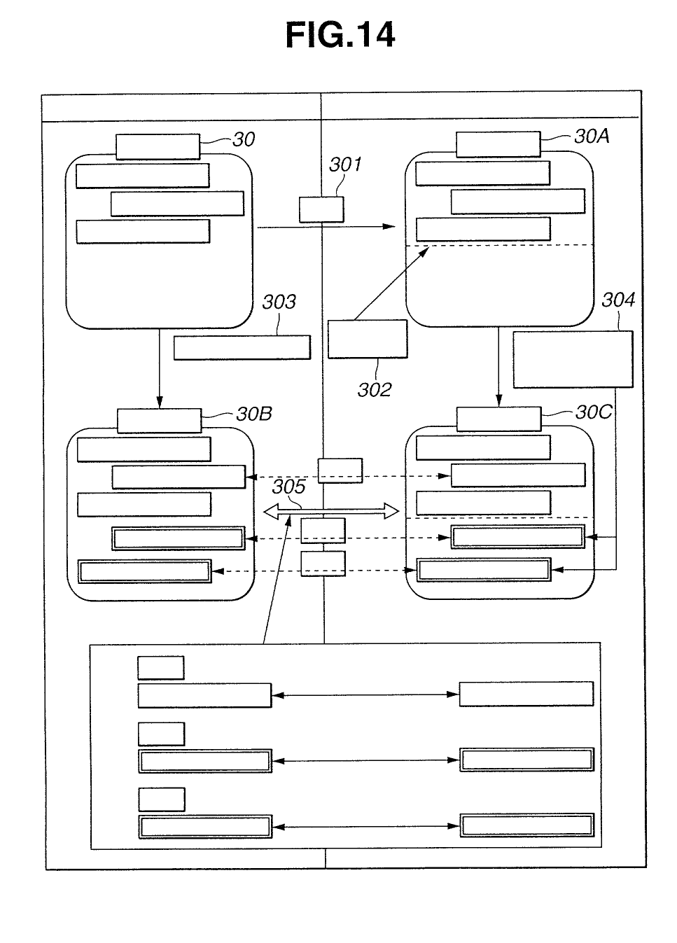

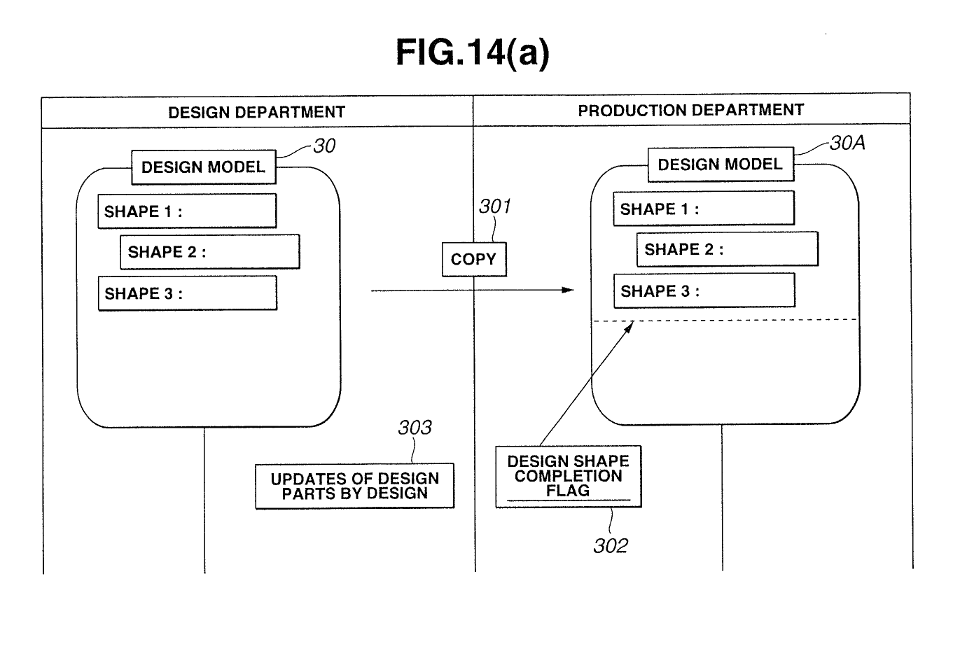

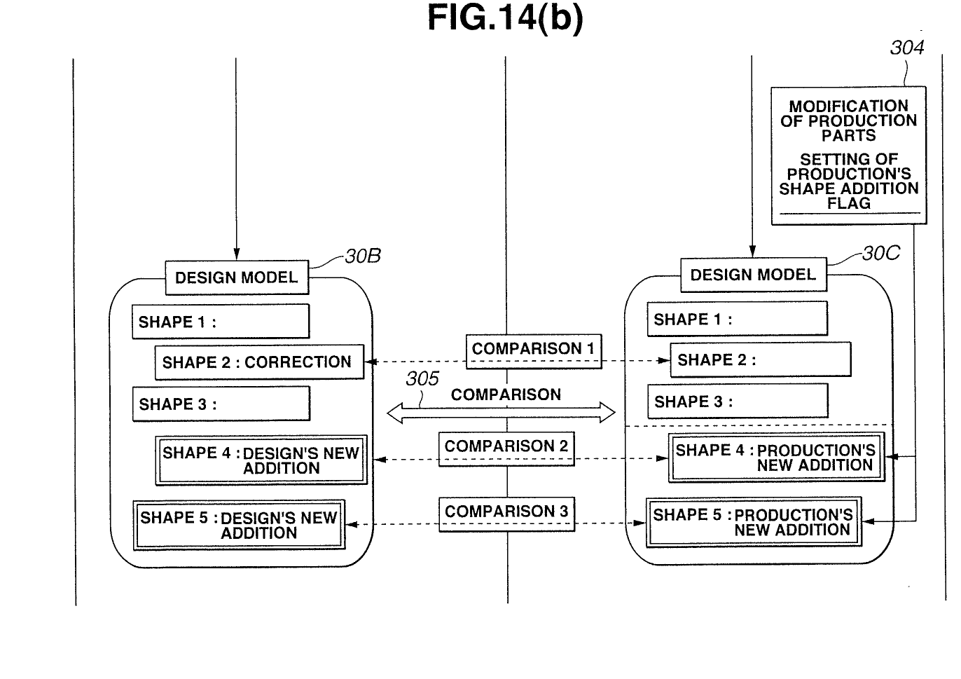

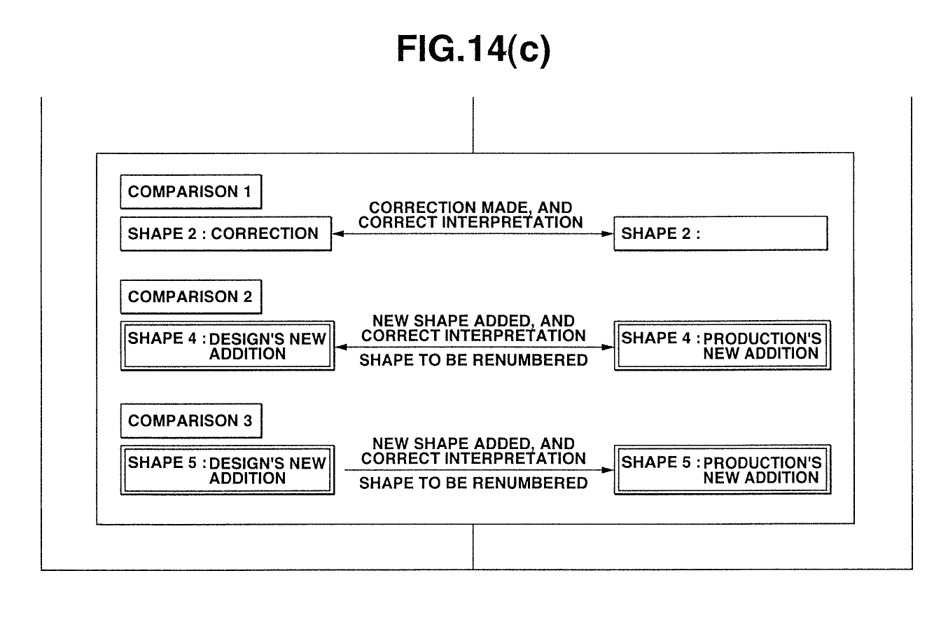

In the embodiment described above, the part of the three-dimensional model is corrected by Production Department. The same procedure can also be applied when the part of the three-dimensional model is corrected by Design Department as shown in FIGS. 14(a) to 14(c), which are parts of FIG. 14.

Second Embodiment:

As shown in FIGS. 14(a) to 14(c), the original three-dimensional model 30 created by Design Department is copied by Production Department (step 301). FIG. 1 shows perspectively the original three-dimensional model 30 created by Design Department. FIG. 2 shows perspectively the three-dimensional model 30A copied by Production Department. The three-dimensional models 30,30A are configured by allotting identification ID Nos. 1 to 3 (shape Nos. 1, 2, 3) to the respective component parts and overlaying parts with reference to the already overlaid parts by other parts. The three-dimensional CAD system executes the commands associated with the identification ID Nos. 1 to 3 in order of the identification ID Nos. 1, 2, 3 to reproduce the shapes of the three-dimensional models 30, 30A.

Then, a design shape completion flag is set for the identification ID Nos. 1 to 3 of the parts configuring the copied three-dimensional model 30A (step 302).

Then, a production shape addition flag is set. In other words, the production shape addition flag is given to the identification ID of a part to be added to the three-dimensional model 30A by Production Department (step 304).

Production Department newly adds production shapes having identification ID Nos. 4, 5. In other words, the parts such as the core 34 and the deadheads 36, 37 are added to the three-dimensional model 30A of FIG. 2 to create the new production three-dimensional model 30C as shown in FIG. 4. Identification ID No. 4 is given to the core 34, and identification ID No. 5 is given to the deadheads 36, 37. A production shape addition flag is set for the identification ID Nos. 4, 5.

Meanwhile, Design Department adds respective parts (design shapes) having identification ID Nos. 4, 5 to the original three-dimensional model 30 and corrects dimensions or the like of the part having the identification ID No. 2 to create new three-dimensional model 30B. In other words, the parts such as the draft 31, the round 32 and the round 33 are added to the three-dimensional model 30 of FIG. 1 and a hole diameter of the boss is changed to create the new three-dimensional model 30B as shown in FIG. 3. The draft 31 is formed on the side face of the main body of the bracket. The round 32 is formed on the boss. The round 33 is formed on a ridge line. The draft 31 is given the identification ID No. 4, and the rounds 32, 33 are given the identification ID No. 5. Identification ID No. 2 is given to the hole of the boss (step 303).

The design updated three-dimensional model 30B which has the parts added or corrected by Design Department is copied by Production Department.

Then, a design shape completion flag is set for the identification ID Nos. 1 to 5 of the parts configuring the copied three-dimensional model 30B in the same way as in step 302.

Then, the design updated three-dimensional model 30B created by Design Department and the production three-dimensional model 30C created by Production Department are compared for each and same identification ID, and it is judged from the flag given to the same identification ID whether the part is corrected or added.

In other words, the design shape completion flag is given to the identification ID No. 2 for the design updated three-dimensional model 30B and the production three-dimensional model 30C. The part having the identification ID No. 2 has its dimensions changed by Design Department, so that the details of the part are different between the design updated three-dimensional model 30B and the production three-dimensional model 30C. Thus, when logic, “there are a design shape completion flag and a content difference” is approved, the part having the identification ID No. 2 is judged as “corrected”.

Meanwhile, a design shape completion flag is given to the identification ID Nos. 4, 5 of the design updated three-dimensional model 30B, and a production shape addition flag is given to the identification ID Nos. 4, 5 of the production three-dimensional model 30C. The parts having the identification ID Nos. 4, 5 are separately added by Design Department and Production Department, so that the details of the parts are different between the design updated three-dimensional model 30B and the production three-dimensional model 30C. Thus, when logic, “there are a design shape completion flag, a production shape addition flag and a content difference” is approved, the parts having the identification ID Nos. 4, 5 are recognized as “added”.

Therefore, the identification ID is renumbered so that the design updated three-dimensional model 30B and the production three-dimensional model 30C have a different identification ID. In other words, the identification ID Nos. 4, 5 of the production three-dimensional model 30C are renumbered to identification ID Nos. 16, 17 (step 305).

Thus, the contents added to the production three-dimensional model 30C can be taken into the design updated three-dimensional model 30B to integrate both the models.

The integrating process is performed in the same way as described with reference to FIG. 5 to FIG. 9.

As described above, the embodiment is configured so that when the three-dimensional model created by Design Department is copied, the design shape completion flag (a first flag) is given to the identification ID of the part configuring the three-dimensional model, and after the three-dimensional model created by Design Department is copied, the production shape addition flag (a second flag) is given to the identification ID of the part to be added by Production Department to the copied three-dimensional model, so that it can be recognized clearly whether the parts having the same identification ID are corrected or added. Therefore, it becomes possible to integrate the three-dimensional models concurrently processed and created by Design Department and Production Depart, and a lead time can be reduced considerably.

In the embodiment described above, the same design shape completion flag is given to the three-dimensional models 30, 30B created by Design Department when they are copied by Production Department. It becomes possible to make detailed judgment about correction and addition by addition of a different flag every time the three-dimensional model created by Design Department is copied by Production Department.

Third Embodiment:

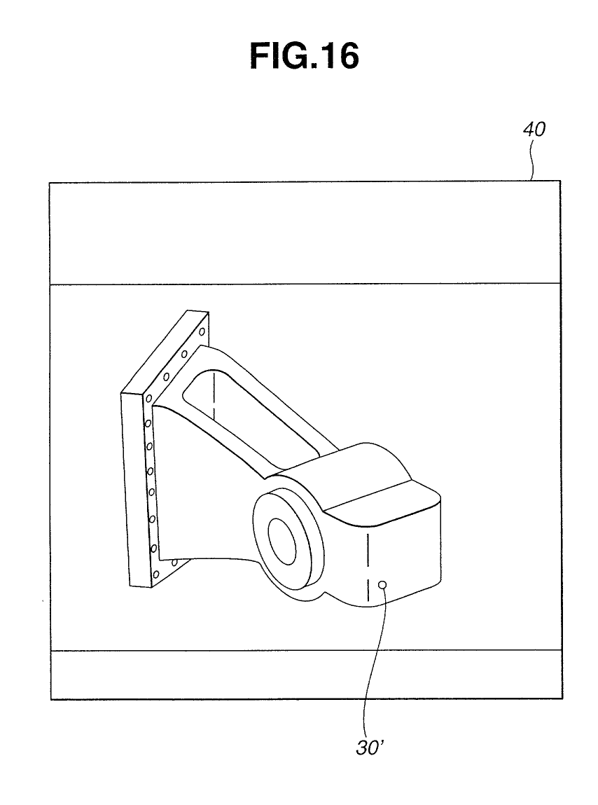

It is assumed in this embodiment that three-dimensional model 30′ of a bracket for a construction machine is created on the three-dimensional CAD system as shown in FIG. 16.

FIG. 16 to FIG. 21 show the contents of the display screen 40 of a personal computer disposed in Design Department and Production Department.

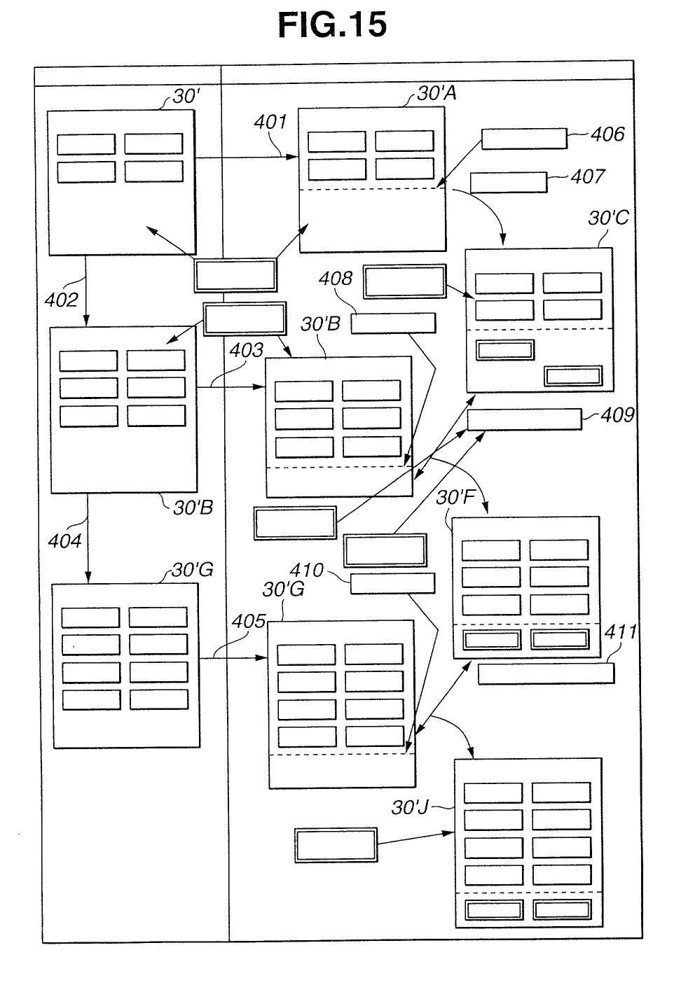

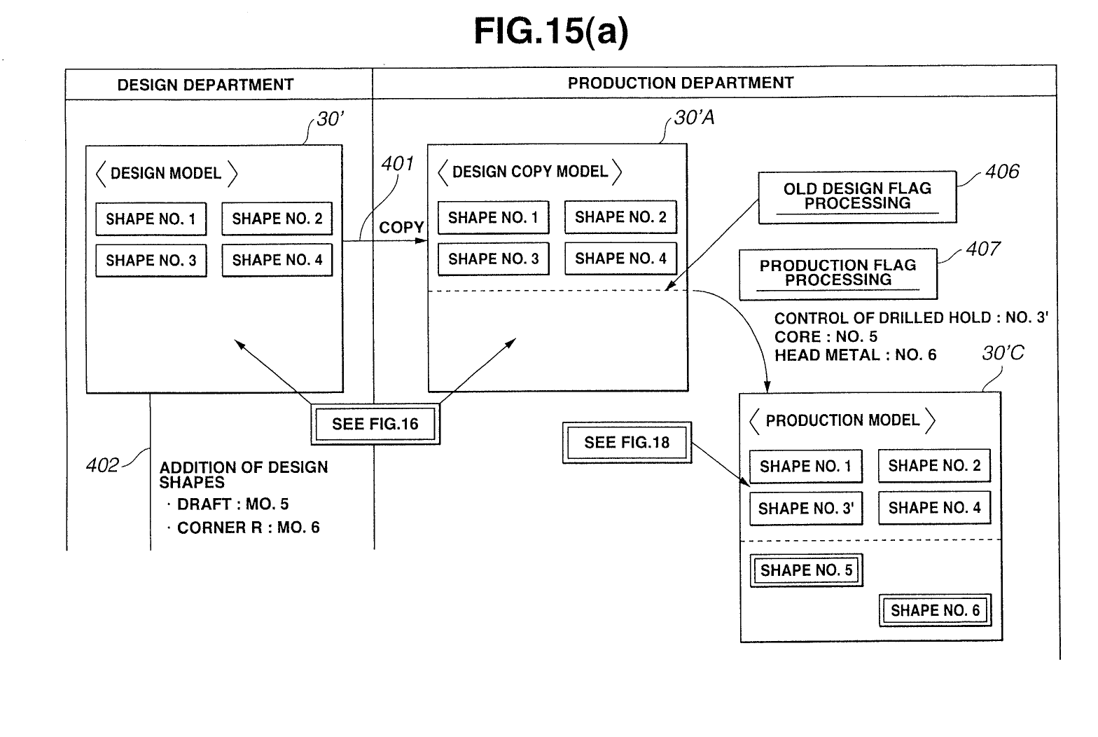

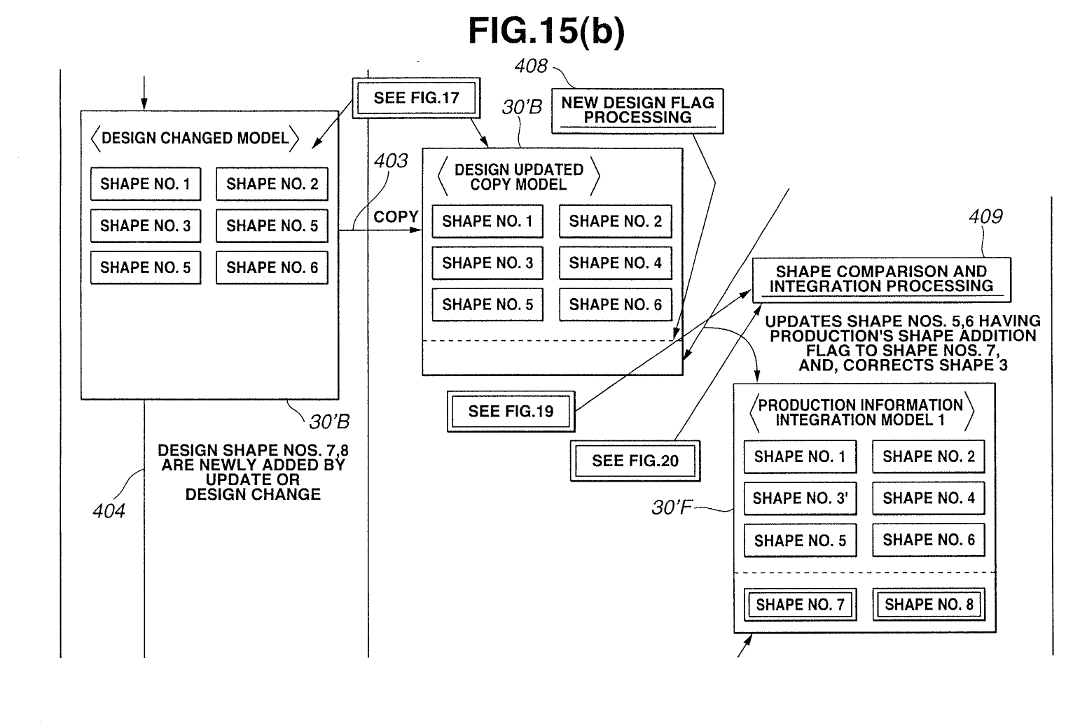

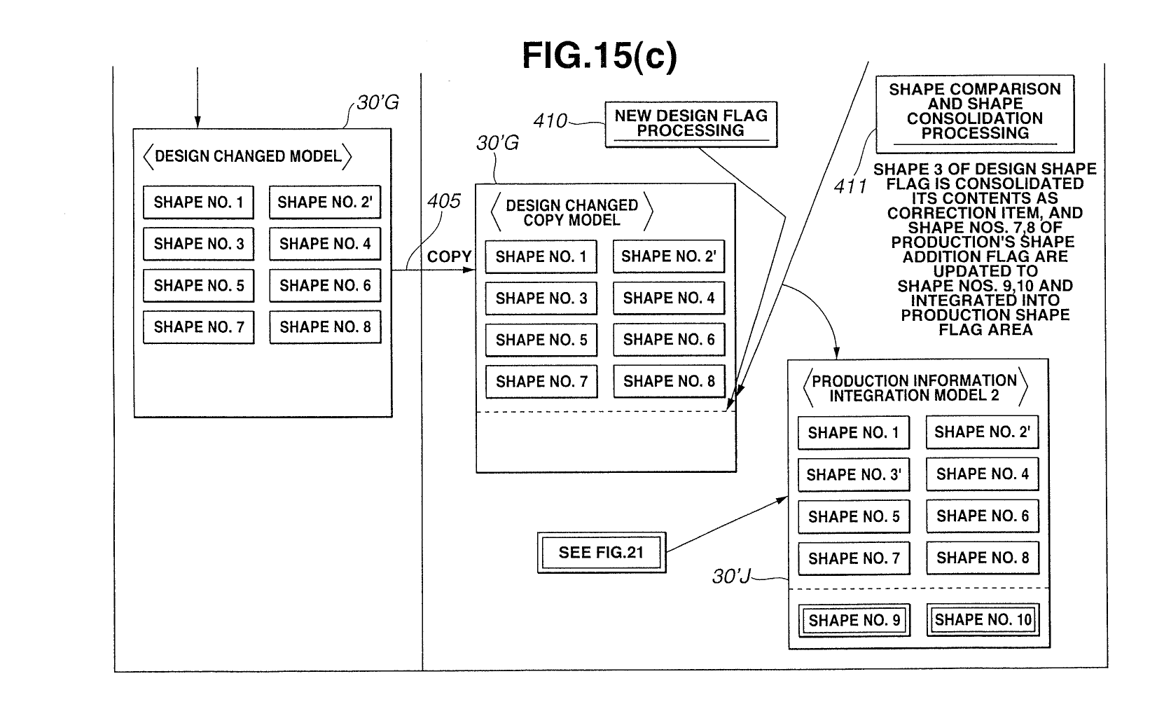

FIGS. 15(a) to 15(c), which are parts of FIG. 15, are flow charts schematically showing a processing procedure of the embodiment. FIGS. 15(a) to 15(c) show a flow of an operation to finally integrate the three-dimensional model by adding and correcting a part with respect to the original three-dimensional model 30′ which is configured of parts having identification ID Nos. 1 to 4 (shape Nos. 1, 2, 3, 4).

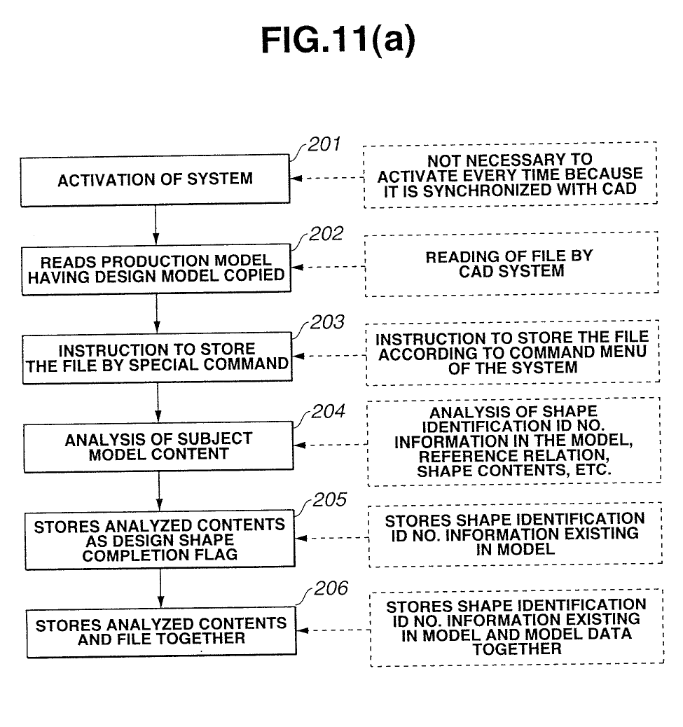

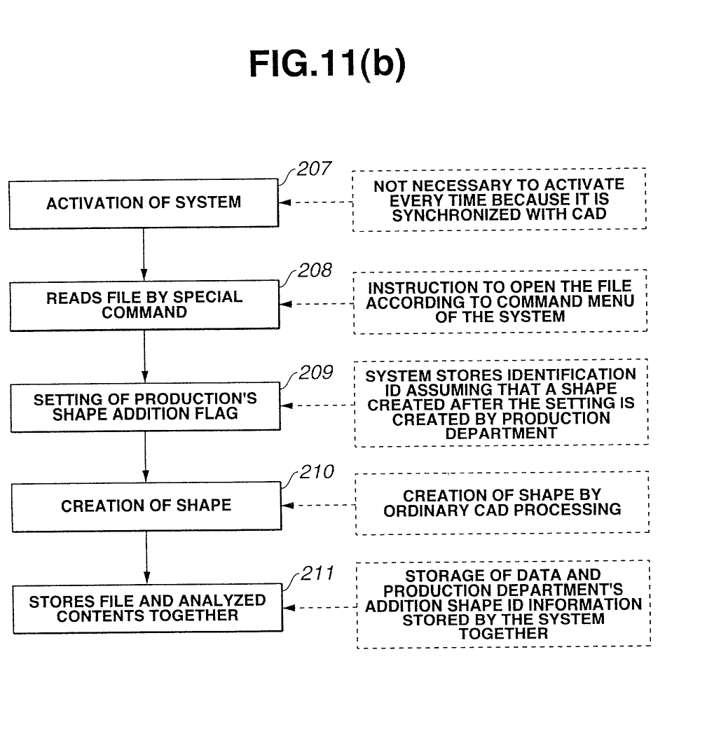

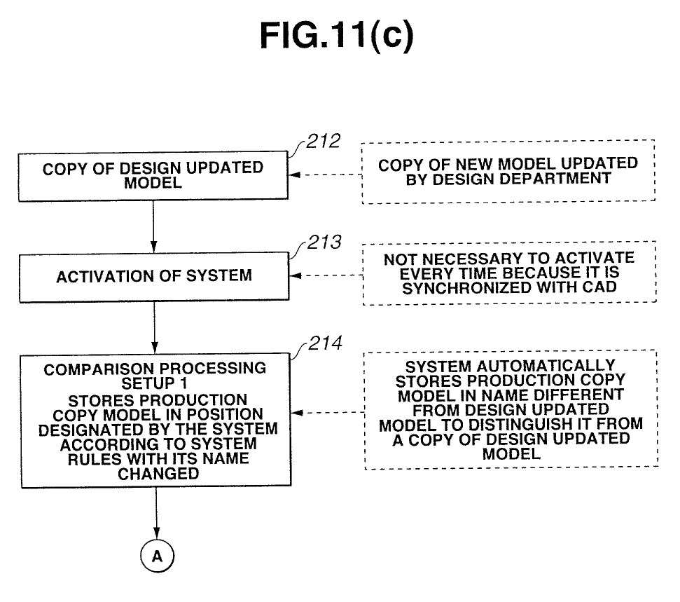

FIGS. 11(a) to 11(c), FIGS. 12(a) to 12(c) and FIG. 13 show a detailed procedure of the process of this embodiment.

Description will be made with reference to these drawings.

As shown in FIGS. 15(a) to 15(c), the original three-dimensional model 30′ created by Design Department is copied by Production Department. The copied three-dimensional model is designated as 30′A (step 401). FIG. 16 shows perspectively the original three-dimensional model 30′ created by Design Department. To configure the three-dimensional model 30′, identification ID Nos. 1 to 4 are allotted to the component parts of the three-dimensional model 30′, and the already overlaid parts are referenced by other parts so to overlay the parts. The three-dimensional CAD system executes commands associated with the identification ID Nos. 1 to 4 in order of identification ID Nos. 1, 2, 3, 4 to reproduce the shape of the three-dimensional model 30′.

Then, an old design flag is set for the identification ID Nos. 1 to 4 of the parts configuring the copied three-dimensional model 30′A (step 406).

FIG. 11(a) shows details of processing 1 to set a design flag (corresponding to the design shape completion flag of FIGS. 10(a) to 10(d) and FIGS. 14(a) to 14(c), including the old design flag, the new design flag to be described later, and the latest design flag).

First, the system is activated. It is not necessary to activate every time because it is synchronized with CAD (step 201). The program of the CAD system is activated then, and a file of the production model 30′A copied from the design model 30′ is read (step 202).

Then, it is instructed to store the file by a special command. Specifically, the storage of the file is instructed when a button on the display screen is pressed (step 203).

Then, identification ID No. information (Nos. 1 to 4) on the parts configuring the three-dimensional model 30′A and data about reference relation and shape are analyzed (step 204).

The old design flag is given to the identification ID Nos. 1 to 4 of the parts configuring the three-dimensional model 30′A and stored (step 205).

Analyzed contents are given to the identification ID Nos. 1 to 4 of the parts configuring the three-dimensional model 30′A and the file is stored (step 206).

The processing 1 in the steps 201 to 206 corresponds to the processing in step 102 of FIG. 10(b).

Then, a production flag (corresponding to the production shape addition flags of FIGS. 10(a) to 10(d) and FIGS. 14(a) to 14(c)) is set. In other words, the production flag is given to the identification ID of the part to be added to the three-dimensional model 30′A by Production Department (step 407).

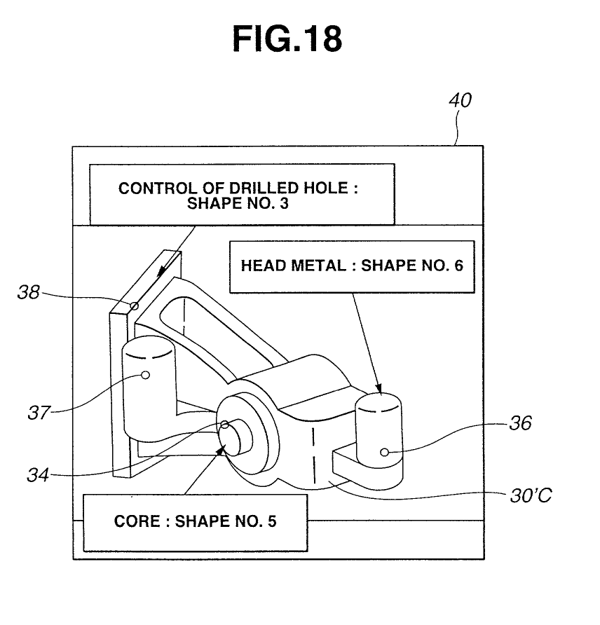

The production shapes of the identification ID Nos. 5, 6 are newly added in Production Department and the shape of the identification ID No. 3 is modified. In other words, the parts such as the core 34 and the deadheads 36, 37 are added to the three-dimensional model 30′A, and machined hole 38 is suppressed to create new production three-dimensional model 30′C as shown in FIG. 18. Identification ID No. 5 is given to the core 34, and identification ID No. 6 is given to the deadheads 36, 37. A production flag is set for the identification ID Nos. 5, 6. As to the machined hole 38 having the identification ID No. 3, correction is made to eliminate it.

FIG. 11(b) shows details of the processing to set the production flag.

The system is activated first. It is not necessary to activate every time because it is synchronized with CAD (step 207). A file of the production model 30′A copied from the design model 30′ is read by a special command (step 208).

Then, processing to set a production flag is executed. Specifically, the flag is set when a button on the display screen is pressed. For the shape created after the flag setting, the system stores the identification ID (No. 4) created when the flag is set, as a shape created by Production Department. Therefore, the production flag is given to the identification ID (No. 5) and later (step 209).

Then, the program of the three-dimensional CAD system is executed, and processing to newly add or correct a shape is executed (step 210).

The analyzed content is then given to the shape recognition ID Nos. 5, 6 of the newly added parts, and a file is stored (step 211).

Processing 2 of the steps 207 to 211 corresponds to that of the steps 103, 104 of FIG. 10(b).

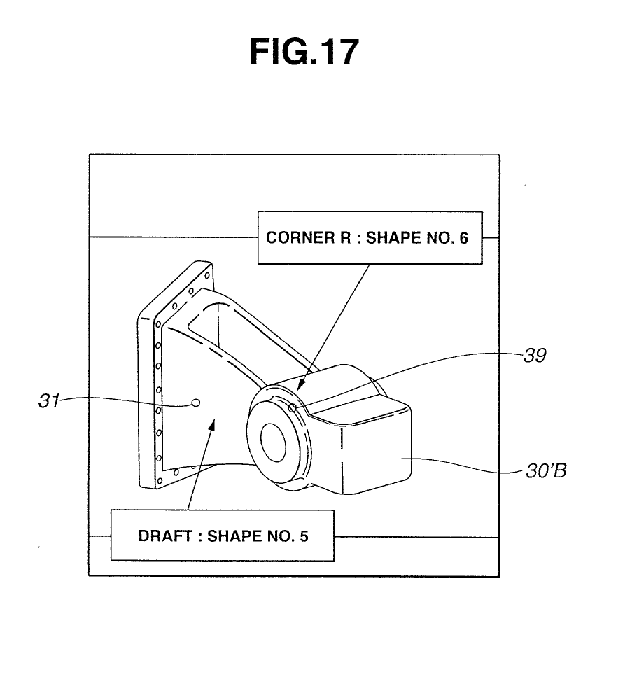

Meanwhile, Design Department adds the respective parts (design shapes) having the identification ID Nos. 5, 6 to the original three-dimensional model 30′ to create the new three-dimensional model 30′B. In other words, parts such as the draft 31 and corner R 39 are added to the three-dimensional model 30 of FIG. 16 to create the new three-dimensional model 30′B as shown in FIG. 17. The draft 31 is formed on the side surface of the bracket body. The corner R 39 is formed on the boss. Identification ID No. 5 is given to the draft 31, and identification ID No. 6 is given to the corner R 39 (step 402).

The design updated three-dimensional model 30′B to which the parts are added by Design Department is copied by Production Department (Step 403).

Then, the design updated three-dimensional model 30′B created by Design Department and the production three-dimensional model 30′C created by Production Department are compared for each and same identification ID, it is judged from the flag given to the same identification ID whether the part is corrected by either of Design Department or Production Department or added by either or both of Design Department and Production Department, and the integrated three-dimensional model is created according to the judged result (step 409).

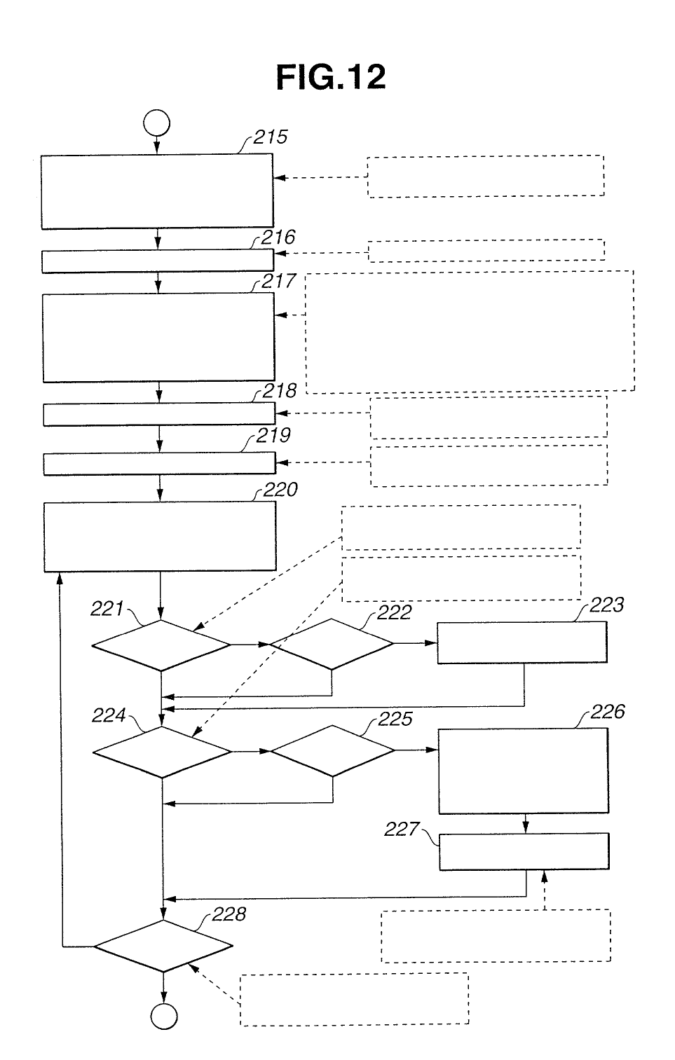

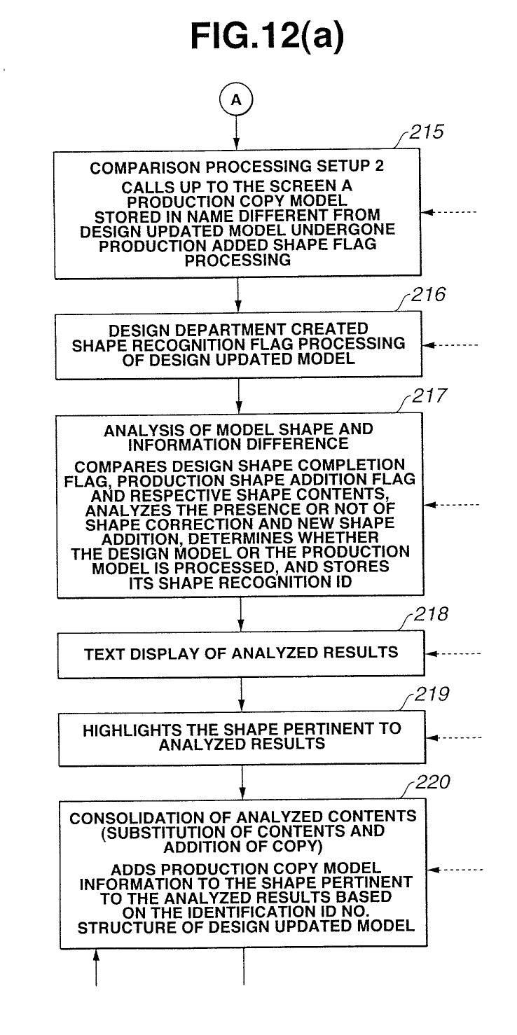

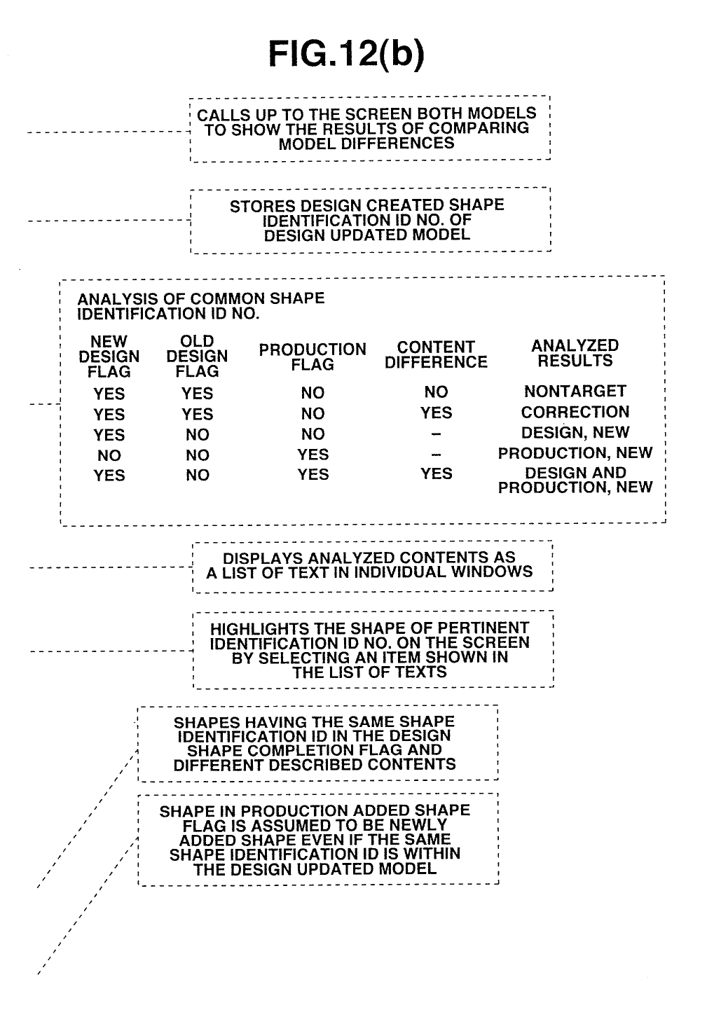

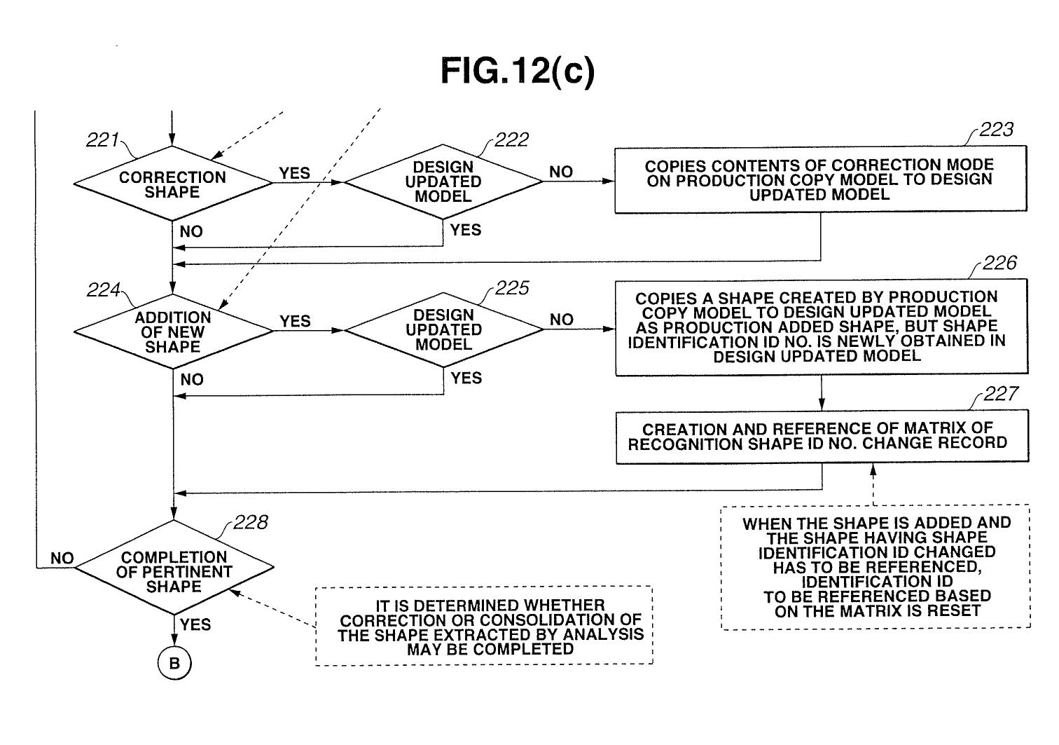

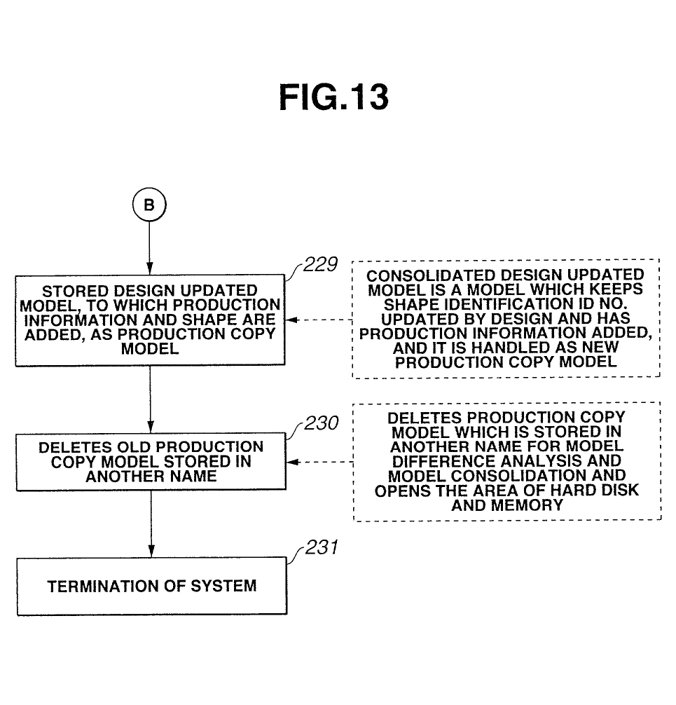

FIG. 11(c), FIGS. 12(a) to 12(c) and FIG. 13 show the details of processing to integrate the shape by comparing the shapes of the three-dimensional models created by both departments.

First, the design updated three-dimensional model 30′B is copied (step 212). Then, the system is activated. It is not necessary to activate every time because it is synchronized with CAD (step 213). In order to distinguish the file of the production three-dimensional model 30′C from the file of the copied design updated three-dimensional model 30′B, the production three-dimensional model 30′C is changed a file name and stored in a designated position of the system according to the system rules (step 214).

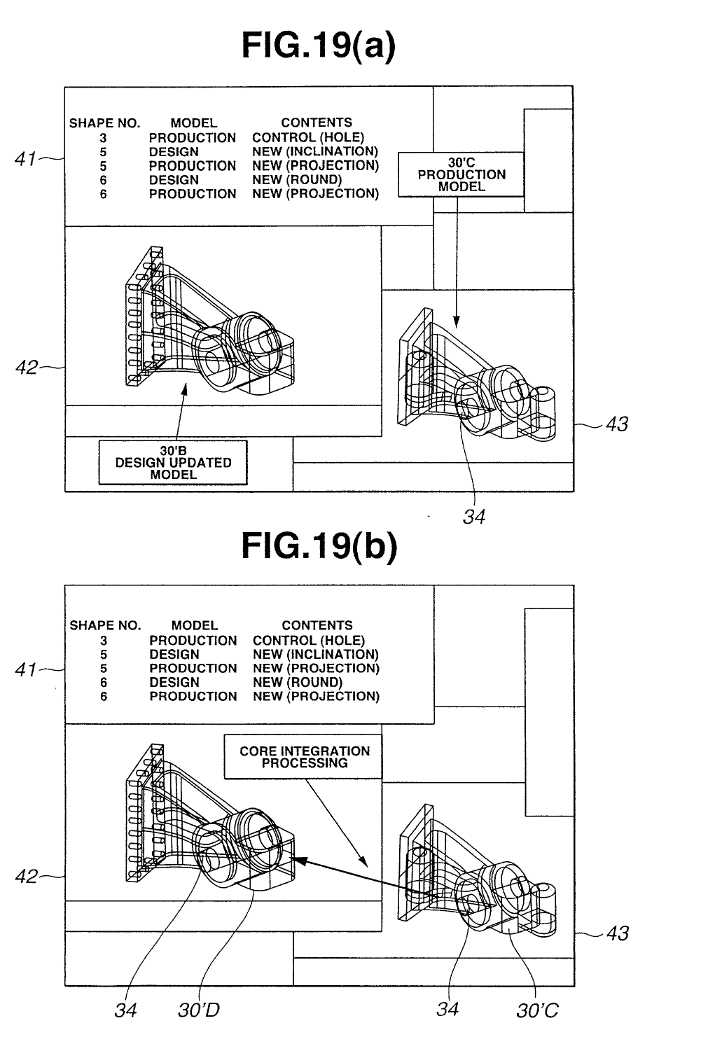

Then, both models 30′B, 30′C are shown on the same screen to compare their differences. As shown in FIG. 19(a), design updated three-dimensional model 30′B is shown in window screen 42 on the display screen 40, and production three-dimensional model 30′C is shown in another window screen 43 (step 215).

A new design flag is set for the identification ID Nos. 1 to 6 of the parts configuring the copied three-dimensional model 30′B (step 216). This processing corresponds to step 408 of FIG. 15(b).

Then, the design updated three-dimensional model 30′B and the production three-dimensional model 30′C are compared for each and same identification ID, and differences in the contents of flag and shape given to the same identification ID are analyzed.

In other words, the new design flag is given to the identification ID Nos. 1, 2, 4 of the design updated three-dimensional model 30′B, the old design flag is given to the identification ID Nos. 1, 2, 4 of the production three-dimensional model 30′C but the production flag is not given. The parts having the identification ID Nos. 1, 2, 4 are not corrected by Design Department or Production Department, so that the contents of the parts are not different between the design updated three-dimensional model 30′B and the production three-dimensional model 30′C. Thus, when logic, “there are a new design flag and an old design flag but not a production flag or a content difference” is approved, the parts having the identification ID Nos. 1, 2, 4 are recognized as “no correction or addition (non-target)”.

And, the new design flag is given to the identification ID No. 3 of the design updated three-dimensional model 30′B, the old design flag is given to the identification ID No. 3 of the production three-dimensional model 30′C but the production flag is not given. The part having the identification ID No. 3 is corrected by Production Department, so that the contents of the parts are different between the design updated three-dimensional model 30′B and the production three-dimensional model 30′C Thus, when logic, “there are a new design flag and an old design flag but not a production flag or a content difference” is approved, the part having the identification ID No. 3 is recognized as “corrected”.

The new design flag is given to the identification ID Nos. 5, 6 of the design updated three-dimensional model 30′B, and the old design flag is not given to but the production flag is given to the identification ID Nos. 5, 6 of the production three-dimensional model 30′C.

The parts having the identification ID Nos. 5, 6 are separately added by Deign Department and Production department, so that the contents of the parts are different between the design updated three-dimensional model 30′B and the production three-dimensional model 30′C. Thus, when logic, “there is a new design flag, there is not an old design flag, there is a production flag, and there is a content difference” is approved, the parts having the identification ID Nos. 5, 6 are recognized as “parts newly added by Design Department and Production Department”.

If it is assumed that the part having the identification ID No. 7 is further added by Design Department only, the new design flag is given to the identification ID No. 7 of the design updated three-dimensional model 30′B, the production three-dimensional model 30′C does not have a corresponding identification ID No. 7 and either the old design flag or the production flag is not given. Thus, when logic, “there is a new design flag, there is not an old design flag, and there is not a production flag” is approved, the part having the identification ID No. 7 is recognized as “part newly added by Design Department”.

Conversely, when it is assumed that the part having the identification ID No. 7 is added by Production Department only, an old design flag is not given to but a production flag is given to the identification ID No. 7 of the production three-dimensional model 30′C, the design updated three-dimensional model 30′B does not have a corresponding identification ID No. 7 and a new design flag is not given to it. Thus, when logic, “there is not a new design flag, there is not an old design flag, and there is a production flag” is approved, the part having the identification ID No. 7 is recognized as “part newly added by Production Department” (step 217).

Then, the analyzed result is shown in text form such as characters, numerals and others on the window screen 41 in the display screen 40 as shown in FIG. 19(a). This window screen 41 shows a “model” and a “content” for each “shape No. (identification ID)”. The “model” indicates a distinction between Production Department and Design Department. The “content” indicates a distinction between the corrected content and the added shape. For example, when “shape No. (identification ID)”, a “model” and a “content” are shown as “3”, “projection” and “suppression (hole)”, the operator can know, “the hole having the identification ID 3 was corrected as it was suppressed by Production Department”. When “shape No. (identification ID)”, a “model” and a “content” are shown as “5”, “design” and “new (inclination)” and also “5”, “production” and “new (projection)”, the operator can know, “the identification ID 5 is newly added by Design Department and Production Department, and the added contents are different between the inclination and the projection” (step 218).

Then, the displayed text in the window screen 41 is clicked or the like to select an item to highlight the shape having the pertinent identification ID in the window screens 42, 43. For example, when the operator clicks the displayed text, “5 Production New (Projection)” in the window screen 41, the core 34 having corresponding identification ID No. 5 is highlighted in color distinguishable from other shapes in the window screen 43 (step 219).

Then, the operator compares the window screens 42 and 43 to designate a type of part to be consolidated in the window screen 43.

The identification ID of the designated part is judged according to the analyzed results of step 217 whether the identification ID is renumbered or not and consolidated (steps 221 to 228).

It is judged in step 221 whether the new design flag and the old design flag are given to the same identification ID and the contents are different, namely whether it is a corrected shape or not. In step 222, it is judged whether the design updated model 30′B has the corrected shape. In step 224, it is judged whether the old design flag is given to the identification ID, namely whether a new shape is added. In step 225, it is judged whether the design updated model 30′B has an added shape.

When the core 34 having the identification ID No. 5 is instructed in the window screen 43, it is judged NO in step 221, it is judged YES in step 224, and it is judged NO in step 225. As a result, it is processed in step 226 that the identification ID No. 5 is renumbered to No. 7 so that the design updated three-dimensional model 30′B and the production three-dimensional model 30′C have a different identification ID. Processing in step 227 will be described later. Therefore, as shown in FIG. 19(b), the shape of the core 34 added to the production three-dimensional model 30′C is taken into the design updated three-dimensional model 30′B in the window screen 42 to create new design updated three-dimensional model 30′D (step 228).

Thus, every time the integration processing of the pertinent shape is completed (judged NO in step 228), the procedure is shifted to step 220, and the same processing is repeatedly performed (steps 221 to 227).

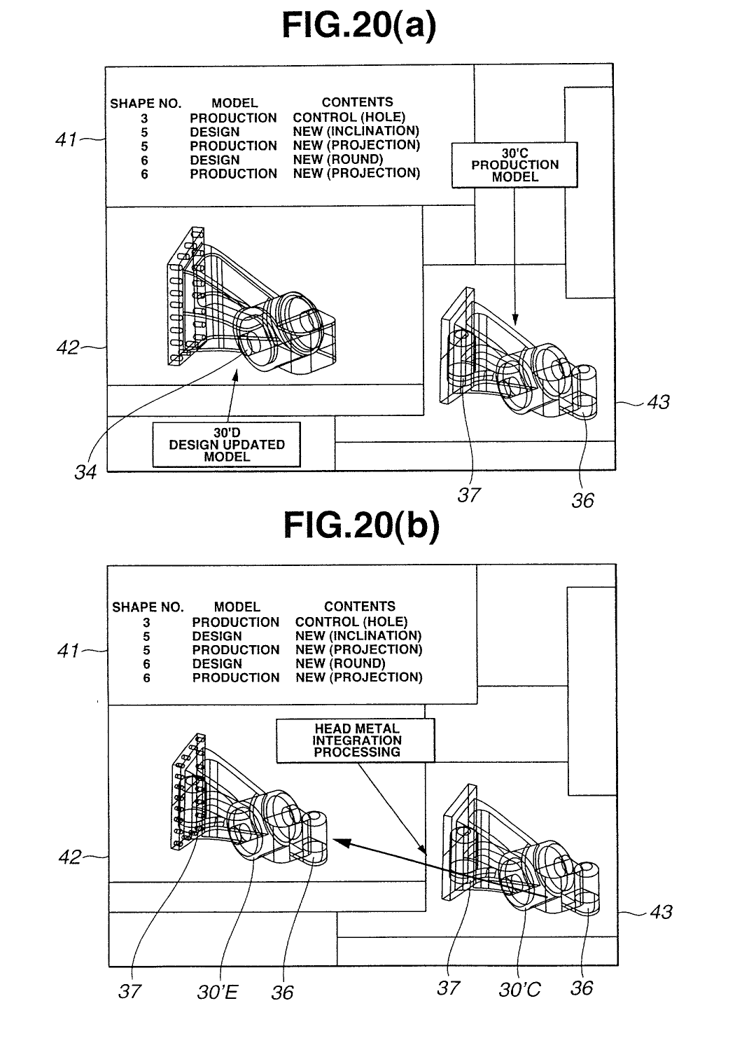

FIG. 20(a) shows display screen 40 before the deadheads 36, 37 are integrated.

When the deadheads 36, 37 having the identification ID No. 6 are instructed in the window screen 43 of FIG. 20(a) in the same way as the integration of the core 34, it is judged NO in step 221, it is judged YES in step 224, and it is judged NO in step 225. As a result, the identification ID No. 6 is renumbered to No. 8 in step 226 so that the identification ID is different between the design updated three-dimensional model 30′D and the production three-dimensional model 30′C. Therefore, the shapes of the deadheads 36, 37 added to the production three-dimensional model 30′C are taken into the design updated three-dimensional model 30′D in the window screen 42 to form new design updated three-dimensional model 30′E as shown in FIG. 20(b) (step 228).

When drilled hole 38 having the identification ID No. 3 is instructed in the window screen 43, it is judged YES in step 221, and it is judged NO in step 222. As a result, suppression of the drilled hole 38 which is a correction content in the production three-dimensional model 30′C is integrated into the design updated three-dimensional model 30′E in step 223 (step 223).

When the shapes of the identification ID Nos. 1, 2, 4 are instructed in the window screen 43 of FIGS. 20(a) and 19(b), it is judged NO in step 221, it is judged NO in step 224, and integration processing is not performed.

When the integration processing of all the shapes is completed (judged YES in step 228), the procedure is shifted to step 229 of FIG. 13, the integrated design updated three-dimensional model 30′E is stored as new production three-dimensional model 30′F (step 229). Then, the old production three-dimensional model 30′C stored with another file name is deleted (step 230), and the processing by the system is terminated (step 231).

Then, it is assumed that addition or correction of parts is made on the three-dimensional model 30′B by Design Department.

As shown in FIGS. 15(a) to 15(c), parts (design shapes) having identification ID Nos. 7, 8 are added to the three-dimensional model 30′B by Design Department, and a part having identification ID No. 2 is corrected to create new three-dimensional model 30′G. It is assumed that the identification ID Nos. 7, 8 are machining allowance. Identification ID No. 2 is a change in dimensions of the part (step 404).

The design updated three-dimensional model 30′G having the parts added or corrected by Design Department is copied by Production Department (step 405).

Then, the design updated three-dimensional model 30′G created by Design Department and the production three-dimensional model 30′F created by Production Department are compared for each and same identification ID. It is judged from the flag given to the same identification ID whether the part is corrected by Design Department or Production Department or added by either Design Department or Production Department or by both of them. And according to the judged result, an integrated three-dimensional model is created (step 411).

Processing of steps 212 to 231 of FIGS. 12(a) to 12(c), which are parts of FIG. 12, and FIG. 13 is executed in the same way.

Specifically, the design updated three-dimensional model 30′G is shown in the window screen 42 of the display screen 40, and the production three-dimensional model 30′F is shown in the window screen 43.

When the core 34 having the identification ID No. 7 is instructed in the window screen 43, it is judged NO in step 221, it is judged YES in step 224, and it is judged NO in step 225. As a result, the identification ID No. 7 is renumbered to No. 9 in step 226 so that the design updated three-dimensional model 30′G and the production three-dimensional model 30′F. have a different identification ID. Therefore, the shape of the core 34 in the production three-dimensional model 30′F is taken into the design updated three-dimensional model 30′G in the window screen 42 to form new design updated three-dimensional model 30′H (step 228).

When the deadheads 36, 37 having the identification ID No. 8 are instructed in the window screen 43 in the same way as the integration of the core 34, it is judged NO in step 221, it is judged YES in step 224, and it is judged NO in step 225. As a result, the identification ID No. 8 is renumbered to No. 10 in step 226 so that the design updated three-dimensional model 30′H and the production three-dimensional model 30′F have a different identification ID. Therefore, the shapes of the deadheads 36, 37 of the production three-dimensional model 30′F are taken into the design updated three-dimensional model 30′H in the window screen 42 to form new design updated model 30′I (step 228).

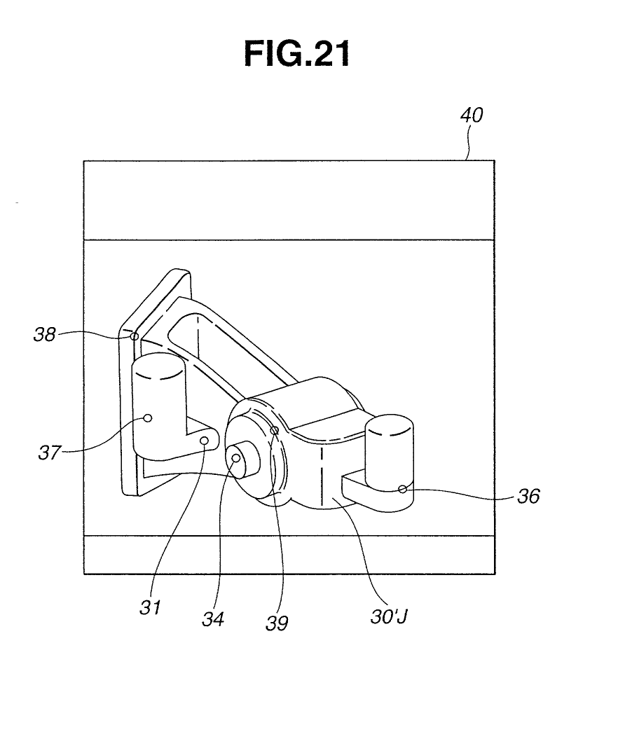

When the integration processing of all shapes is completed (judged YES in step 228), the procedure is shifted to step 229 of FIG. 13, and the integrated design updated three-dimensional model 30′I is stored as new production three-dimensional model 30′J (step 229). Then, the old production three-dimensional model 30′F stored in another name is deleted (step 230), and the processing by the system is terminated (step 231). FIG. 21 shows the three-dimensional model 30′J having undergone the integration processing.

According to the embodiment as described above, the old design flag (first flag) is given to the identification ID of the part configuring the three-dimensional model when the three-dimensional model created by Design Department is copied for a first time, the three-dimensional model created by Design Department is copied, and the production flag (second flag) is given to the identification ID of the part to be added by Production Department to the copied three-dimensional model, and when the third-dimensional model created by Design Department is copied for a second time, new design flag (third flag) is given to the identification ID of the part configuring the three-dimensional model so that it can be recognized clearly that the correction or addition of the pertinent part was made by either Design Department or Production Department. Therefore, the three-dimensional models which were concurrently worked and created by Design Department and Production Department can be integrated, and a lead time is reduced considerably.

It is assumed in the aforementioned embodiment that all the parts added by Production Department are integrated into the three-dimensional model created by Design Department. But, only some of the parts added by Production Department may be integrated into the three-dimensional model created by Design Department.

Fourth Embodiment:

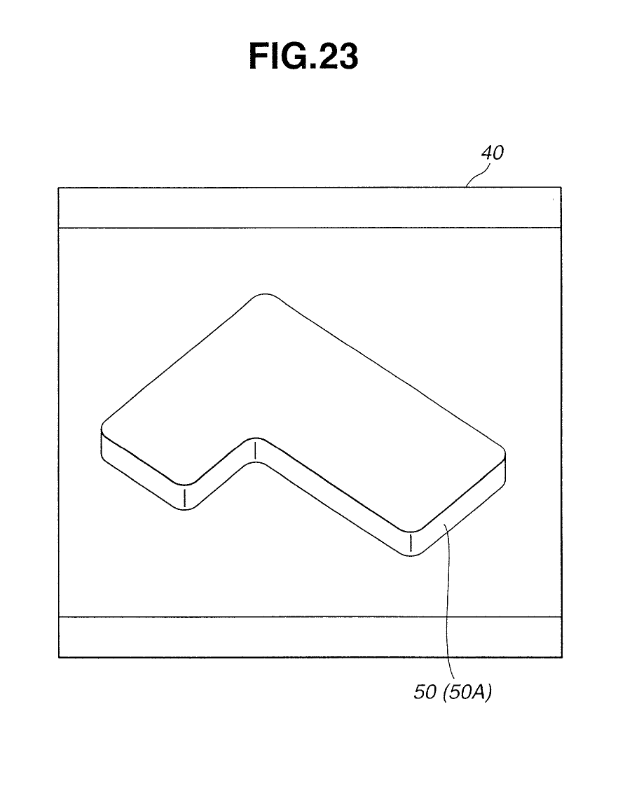

It is assumed in this embodiment that three-dimensional model 50 of a plate is created by the three-dimensional CAD system as shown in FIG. 23.

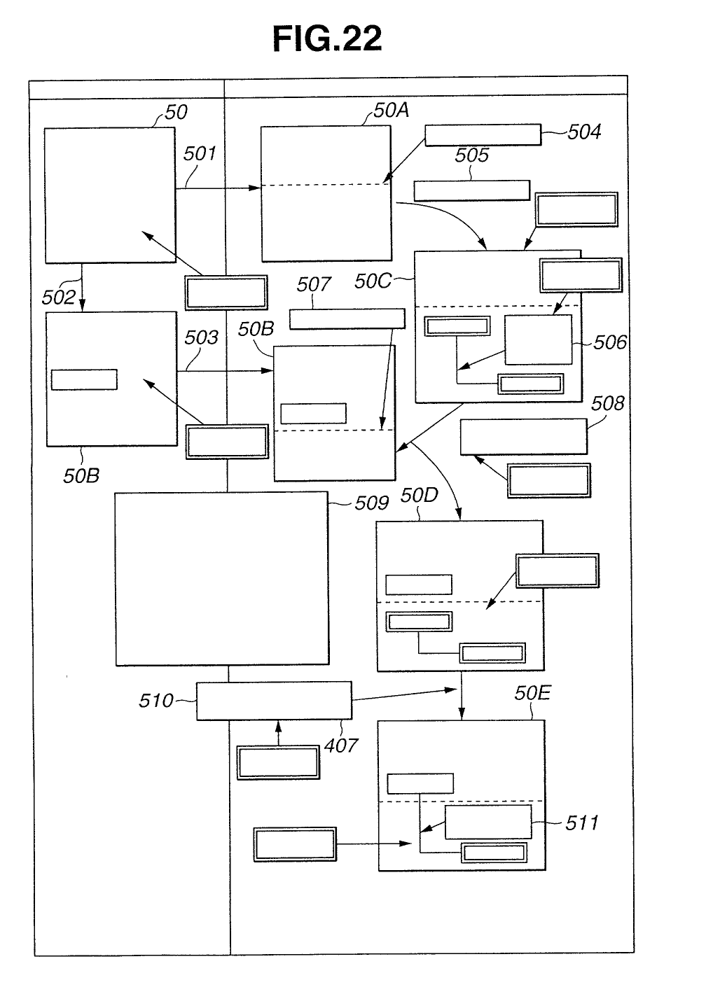

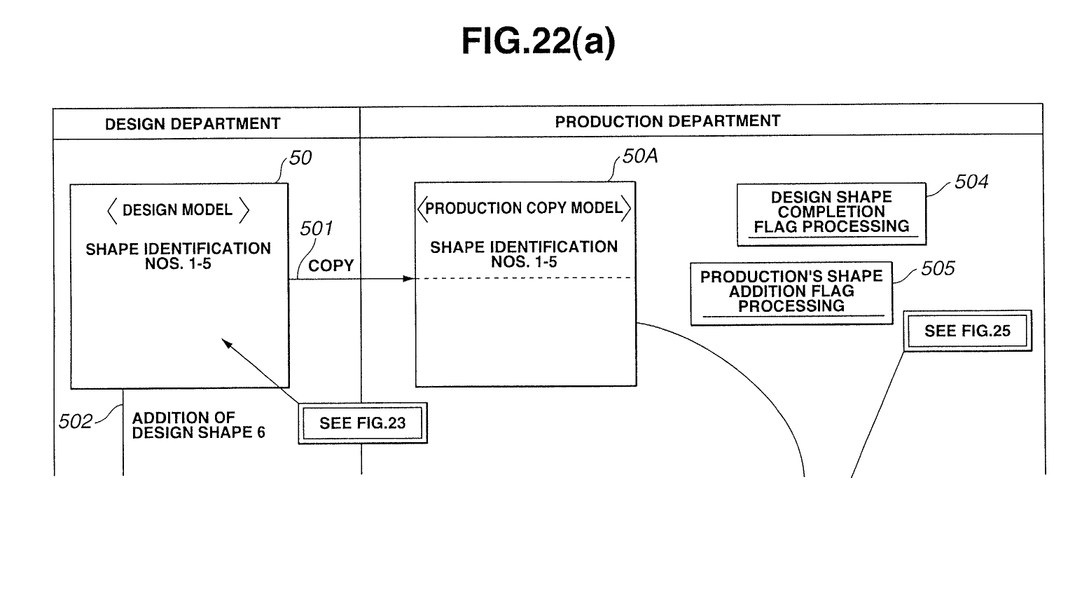

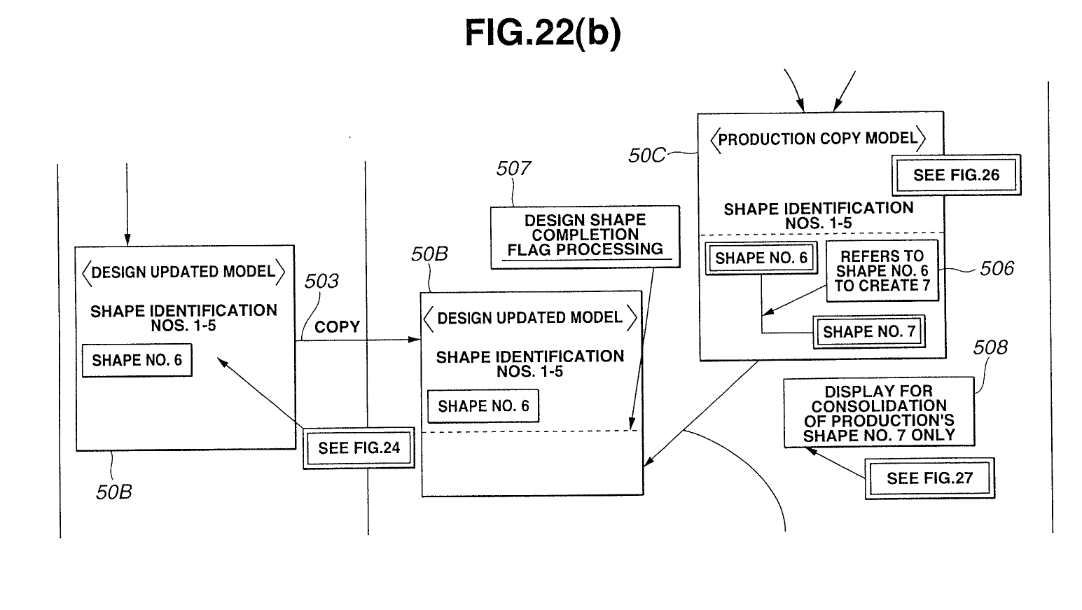

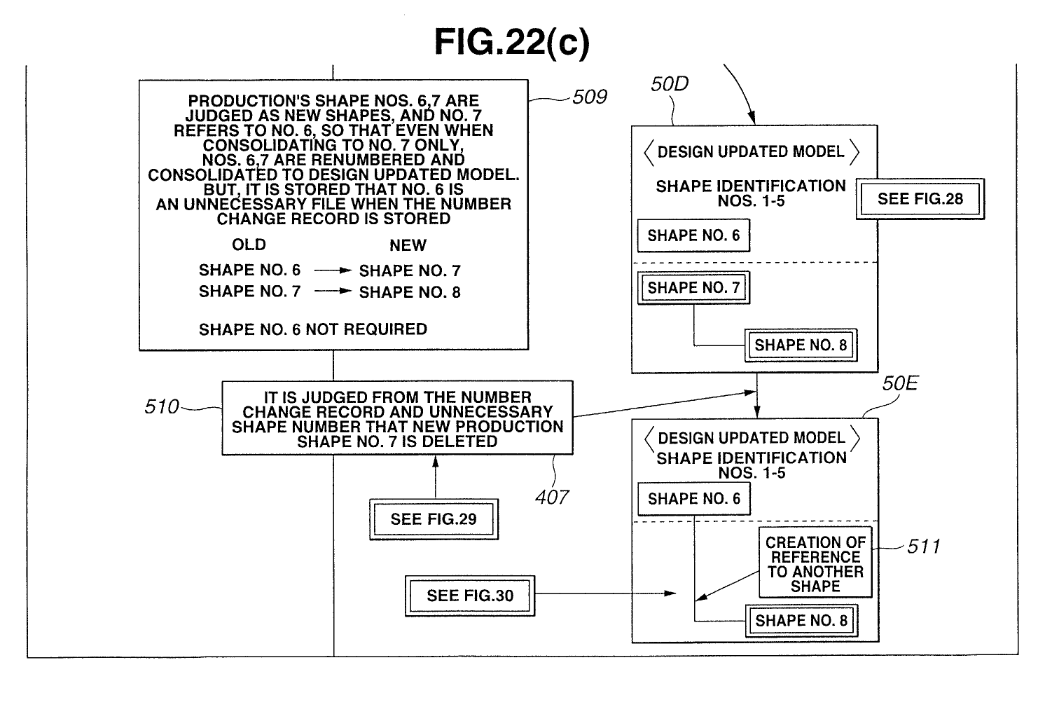

FIG. 23 to FIG. 30 show the contents of the display screen 40 of the personal computer disposed in Design Department and Production Department. FIGS. 22(a) to 22(c), which are parts of FIG. 22, are flow charts schematically showing the procedure of processing of the embodiment.

FIGS. 22(a) to 22(c) show a flow of processing to finally integrate a three-dimensional model by adding parts to original three-dimensional model 50 which is comprised of parts having shape identification Nos. (identification IDs) 1 to 5.

The embodiment will be described with reference to the above drawings.

As shown in FIGS. 22(a) to 22(c), the original three-dimensional model 50 created by Design Department is copied by Production Department. The three-dimensional model copied by Production Department is indicated by 50A (step 501). FIG. 23 perspectively shows the three-dimensional models 50, 50A created by Design Department and copied by Production Department. The three-dimensional models 50, 50A have identification ID Nos. 1 to 5 given to the respective component parts and are configured by referencing the already overlaid parts by other parts to overlay the parts. The three-dimensional CAD system executes commands associated with the identification ID Nos. 1 to 5 in order of the identification ID Nos. 1, 2, 3, 4 and 5 to reproduce the shapes of the three-dimensional models 50, 50A.

Then, a design shape completion flag is set for the identification ID Nos. 1 to 5 of the parts configuring the copied three-dimensional model 50A (step 504).

Then, a production shape addition flag is set. In other words, the production shape addition flag is given to the identification IDs of the parts to be added to the three-dimensional model 50A by Production Department (step 505).

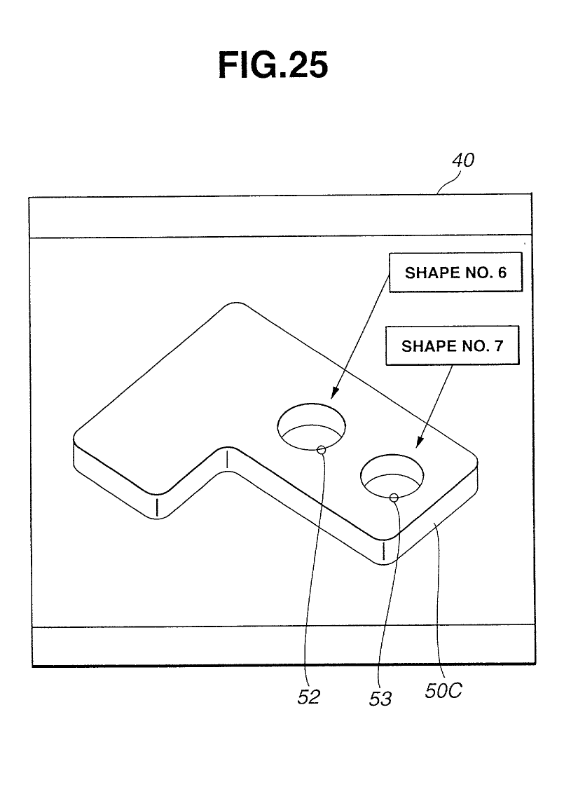

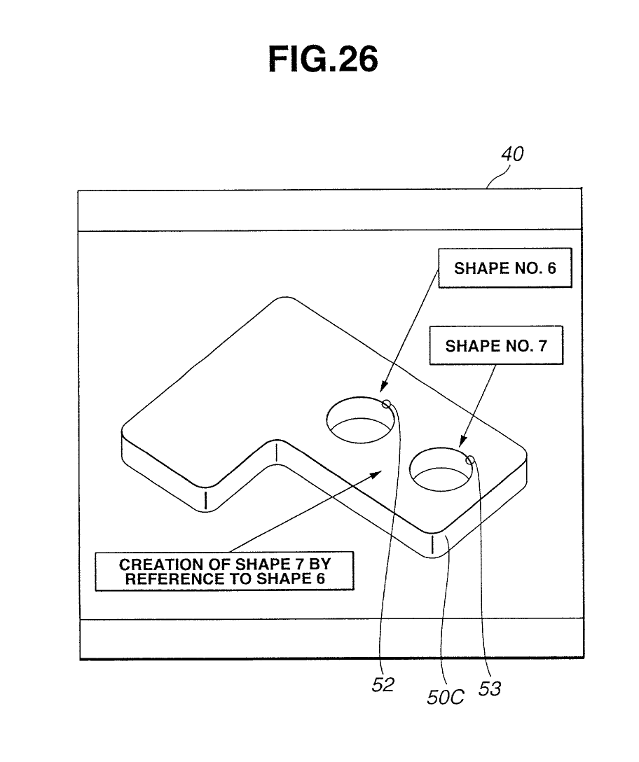

Production Department newly adds production shapes having identification ID Nos. 6, 7. Specifically, parts such as holes 52, 53 are added to the three-dimensional model 50A of FIG. 23 to create new production three-dimensional model 50C as shown in FIG. 25. Identification ID No. 6 is given to the hole 52, and identification ID No. 7 is given to the hole 53. A production shape addition flag is set for the identification ID Nos. 6, 7. As shown in FIG. 26, a command (hole, through) or a parameter (identification IDs 6, d, r) for “making a through hole having a diameter 2 r at a position distance d away from the “hole” 52 identified by the identification ID No. 6” is associated with the hole 53 identified by the identification ID No. 7. In other words, the part as the hole 53 identified by the identification ID No. 7 has relation to the part as the hole 52 identified by the identification ID No. 6 to refer to it (step 506).

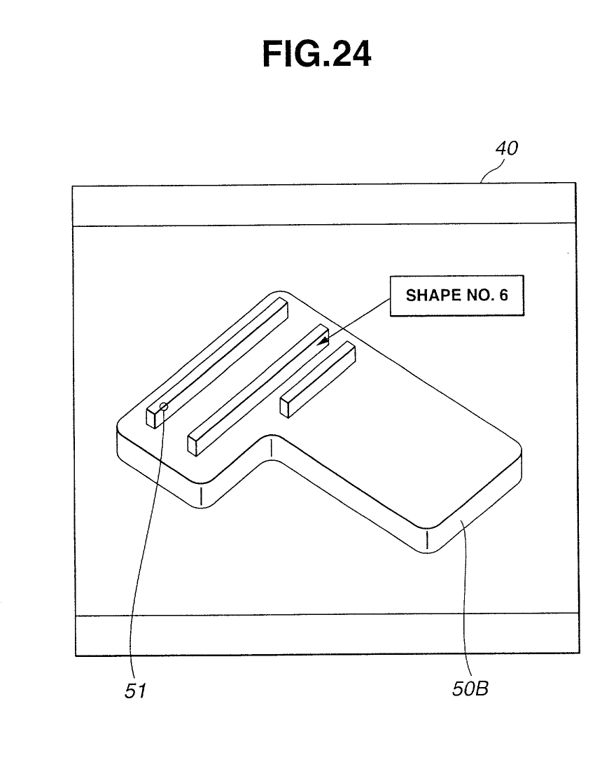

Meanwhile, Design Department adds a part (design shape) having the identification ID No. 6 to the original three-dimensional model 50 to create new three-dimensional model 50B. In other words, a part called rib 51 is added to the three-dimensional model 50 of FIG. 23 to create new three-dimensional model 50B as shown in FIG. 24. Identification ID No. 6 is given to the rib 51 (step 502).

The design updated three-dimensional model 50B having a part added by Design Department is copied by Production Department (step 503).

Then, a design shape completion flag is set for the identification ID Nos. 1 to 6 of the parts configuring the copied three-dimensional model 50B in the same way as in step 504 (step 507).

Then, the design updated three-dimensional model 50B created by Design Department and the production three-dimensional model 50C created by Production Department are compared for each and same identification ID, and it is judged from the flag given to the same identification ID whether the pertinent part was added by either Design Department or Production Department.

Specifically, a design shape completion flag is given to the identification ID No. 6 of the design updated three-dimensional model 50B, and a production shape addition flag is given to the identification ID No. 6 of the production three-dimensional model 50C. The parts with the identification ID No. 6 were separately added by Design Department and Production Department, so that the contents of the parts are different between the design updated three-dimensional model 50B and the production three-dimensional model 50C. Thus, when logic, “there are a design shape completion flag, a production shape addition flag and a content difference” is approved, the parts having the identification ID No. 6 are recognized as “parts added by both of Design Department and Production Department”.

A production shape addition flag is given to the identification ID No. 7 of the production three-dimensional model 50C, and there is not a corresponding identification ID No. 7 for the design updated three-dimensional model 50B. Therefore, the part having the identification ID No. 7 is recognized as “part added by Production Department only”.

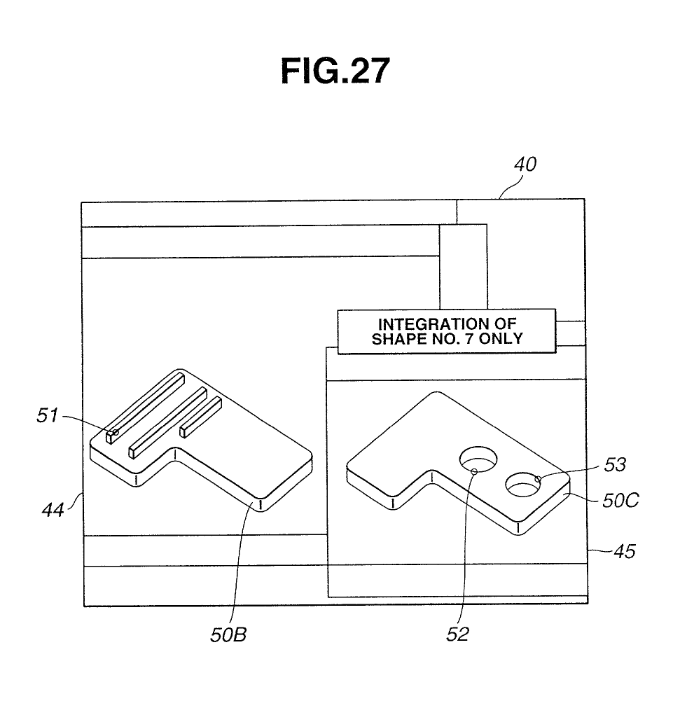

Then, the models 50B and 50C are shown on the same screen to compare them for differences. As shown in FIG. 27, the design updated three-dimensional model SOB is shown in the window screen 44 on the display screen 40, and the production three-dimensional model 50C is shown in another window screen 45.

Then, the operator compares the window screens 44 and 45 to designate a type of part to be consolidated in the window screen 45. In this embodiment, only the hole 53 having the identification ID No. 7 between the holes 52, 53 of the production three-dimensional model 50C shown in the window screen 45 is integrated into the design updated three-dimensional model 50B shown in the window screen 44 (step 508).

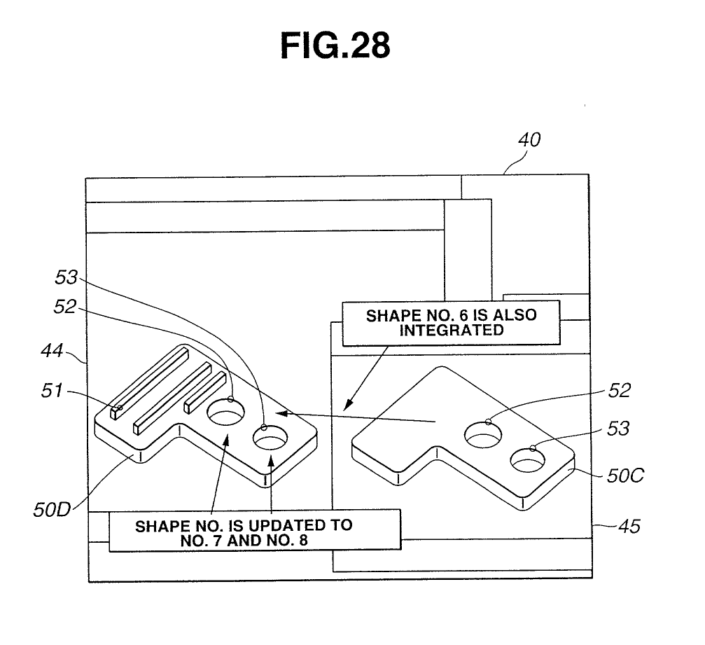

When a command for integrating the identification ID No. 7 only is input to the computer, the identification ID Nos. 6, 7 of the holes 52, 53 are renumbered to Nos. 7, 8, and the holes 52, 53 are consolidated into the design updated three-dimensional model 50B. As a result, the design updated three-dimensional model 50D having the holes 52, 53 consolidated is created on the window screen 44 as shown in FIG. 28. When consolidating, data about nonnecessity of the identification ID No. 6 (hole 52) is stored together with a change record of the identification ID (step 509).

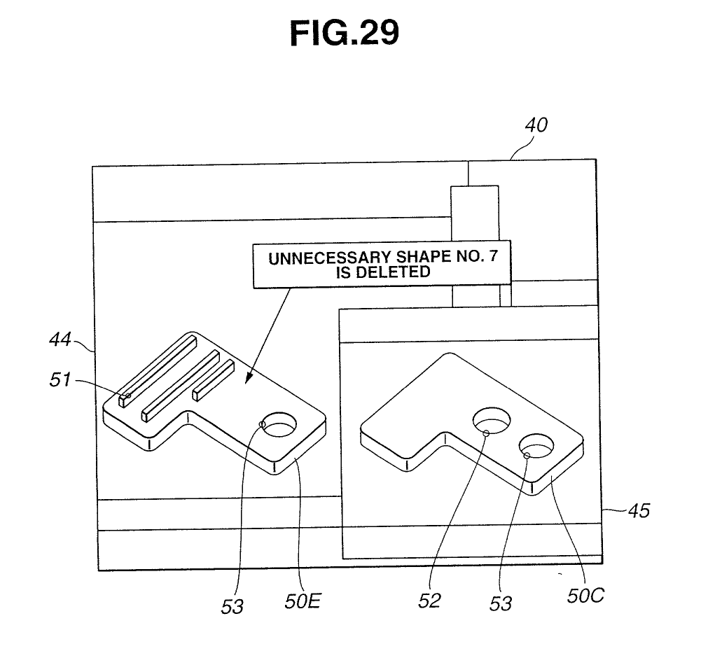

Then, unnecessary identification ID No. 7 (hole 52) is deleted from the design updated three-dimensional model 50D based on storage data that the identification ID Nos. 6, 7 were changed Nos. 7, 8 and storage data that the identification ID No. 6 (hole 52) is unnecessary. As a result, the design updated three-dimensional model 50E from which the hole 52 is deleted is created on the window screen 44 as shown in FIG. 29 (step 510).

Then, a reference relation is changed so that the hole 53 having the identification ID No. 8 references to the part having an identification ID other than the deleted identification ID No. 7.

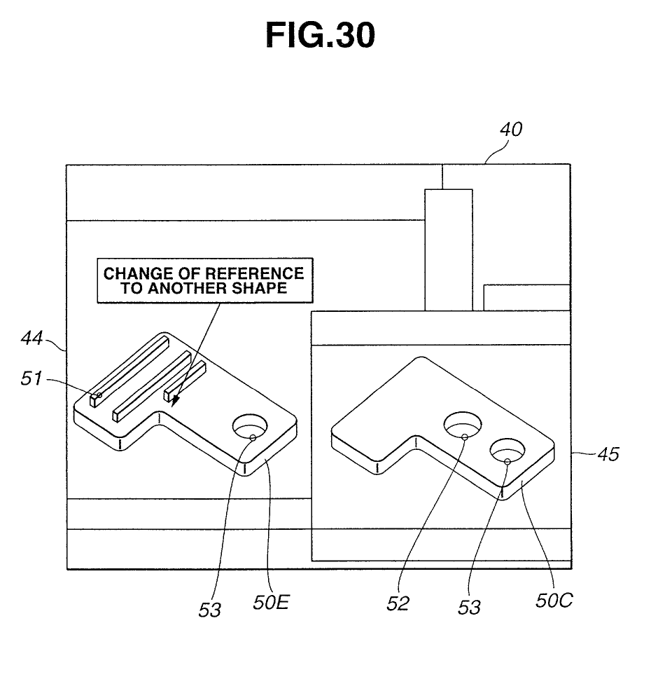

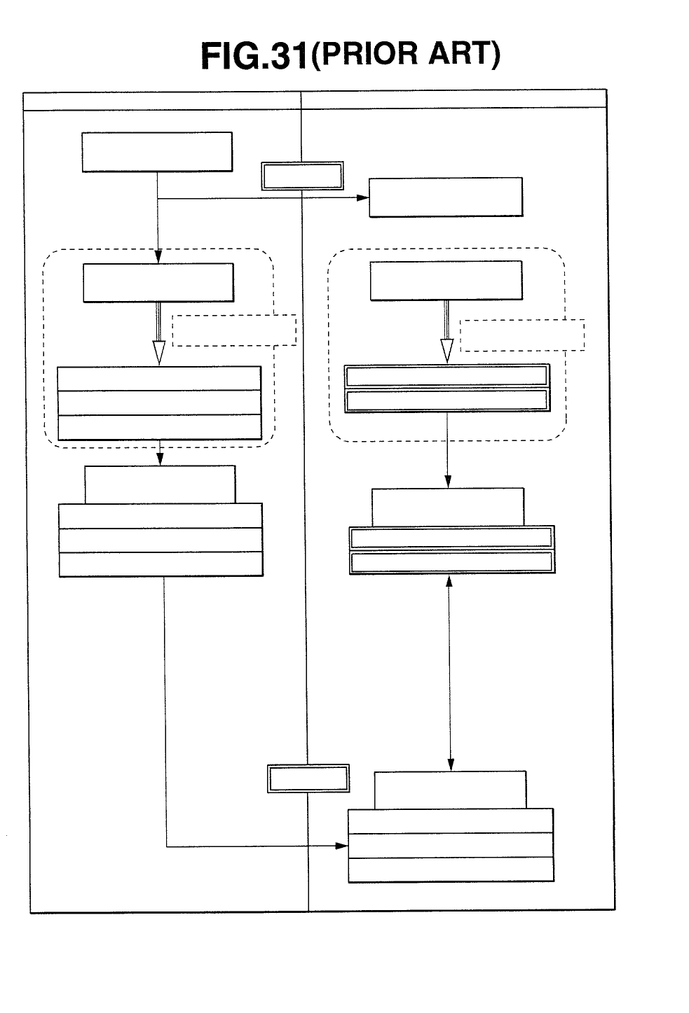

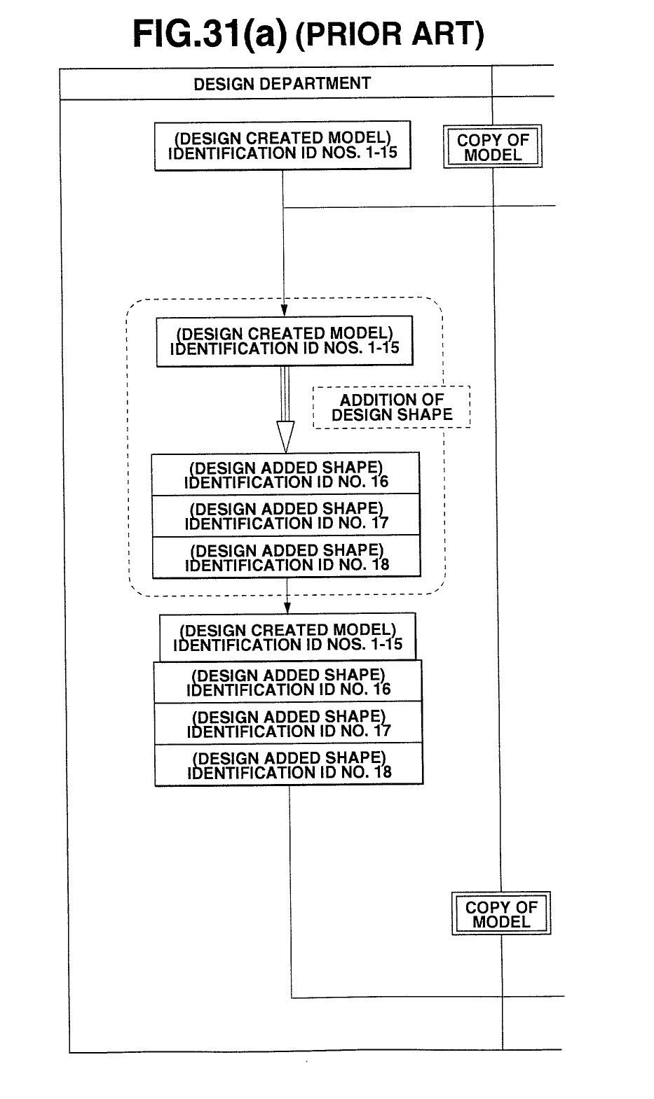

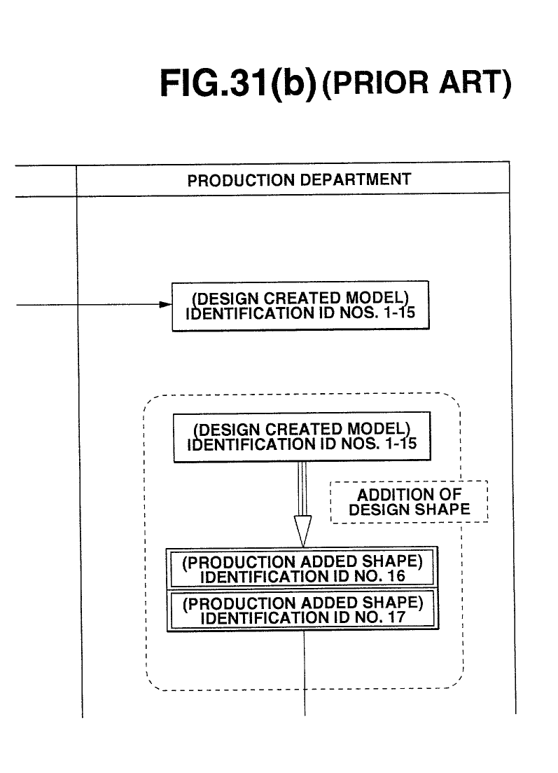

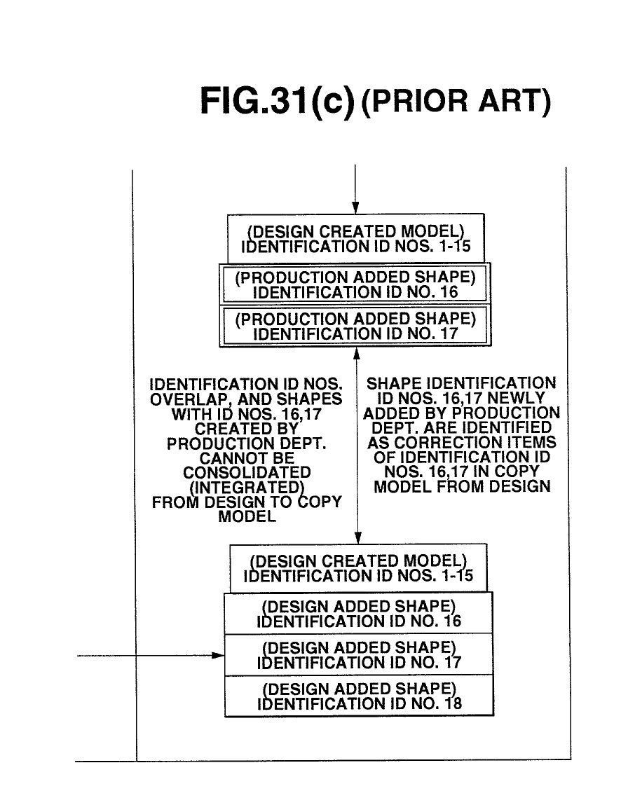

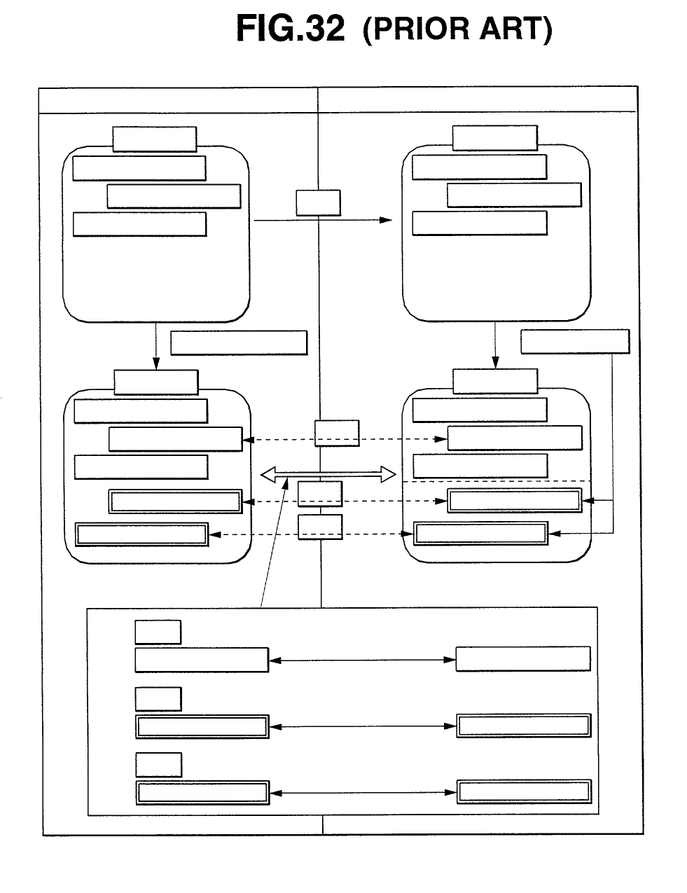

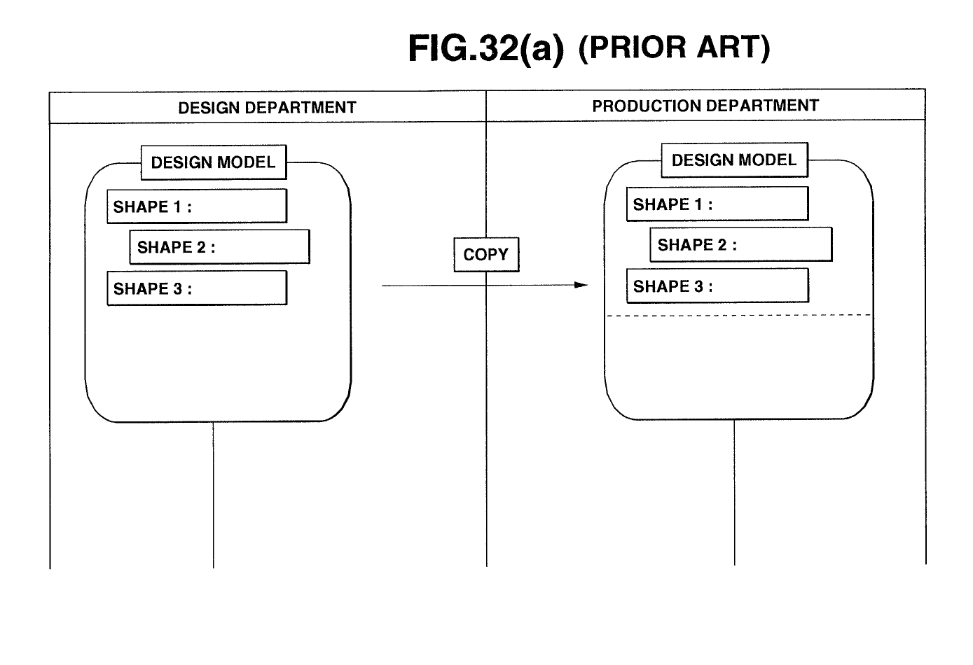

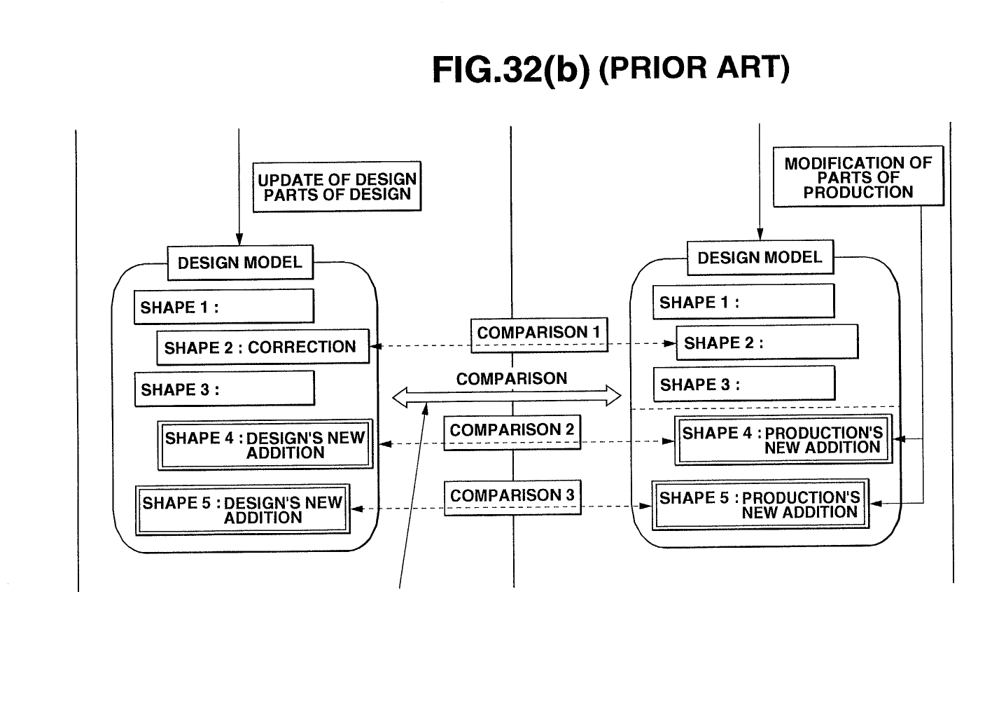

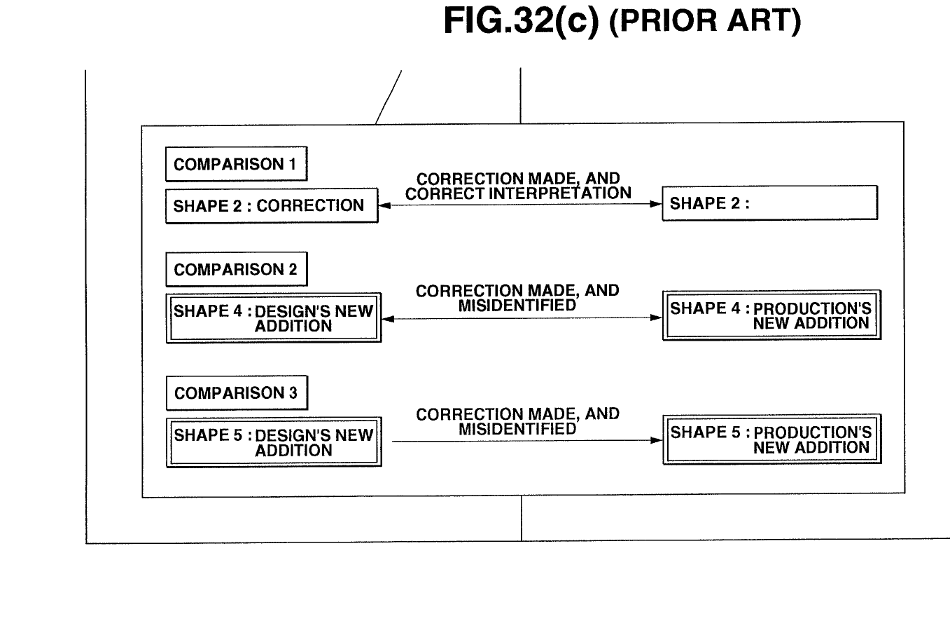

Specifically, a command (hole, through) or a parameter (identification IDs 6, d, r) for “drilling a through hole having a diameter 2 r at a position distance d away from ‘rib’ 51 identified by the identification ID No. 6” is associated with the hole 53 identified by the identification ID No. 8 as shown in the window screen 44 of FIG. 30. In other words, a part called the hole 53 identified by the identification ID No. 8 is changed a reference relation so to refer to the part called the rib 51 identified by the identification ID No. 6. Processing in step 511 corresponds to the processing in step 227 of FIG. 12(c).

According to the embodiment described above, the three-dimensional model 50E which integrates the hole 53 only between the holes 52 and 53 added by Production Department can be created.

According to the embodiment described above, when the hole 53 (first part) configuring the three-dimensional model 50 has a relationship (the identification ID No. 7 refers to identification ID No. 6) to refer to the hole 52 (second part) and only the hole 53 (first part) between the parts is integrated, data that the identification ID (No. 6) of the hole 52 (second part) is unnecessary is stored, the identification IDs of the holes 52, 53 are changed (the identification ID Nos. 6, 7 are changed to Nos. 7, 8), the identification ID (No. 7) of the hole 52 (second part) is deleted based on the stored data, and processing (the identification ID No. 8 refers to the identification ID No. 6) to change the reference relationship is performed so that the hole 53 (first part) refers to another part, the rib 51, so that it becomes possible to integrate only some parts among the plurality of parts added by Production Department into the three-dimensional model created by Design Department.

In the first and second embodiments, when the three-dimensional model created by Design Department is copied, a design shape completion flag (first flag) is given to the identification ID of the part configuring the three-dimensional model, the three-dimensional model created by Design Department is copied, and a production shape addition flag (second flag) is given to the identification ID of the part to be added by Production Department to the copied three-dimensional model.

But, it is also possible to configure that the three-dimensional model created by Design Department is copied, and a production shape addition flag is given to the identification ID of the part to be added by Production Department to the copied three-dimensional model but not to give a design shape completion flag at a time when copied. In this case, when the identification IDs are same and the production shape addition flag is given, it can be judged that the parts are added by Design Department and Production Department, and the identification IDs can be changed according to the judged result so that the identification IDs become different.

What is claimed is:

1. A three-dimensional CAD system which allots identification IDs to respective component parts, references to already overlaid parts to overlay parts so to configure an original three-dimensional model, copies the original three-dimensional model, and concurrently performs addition of parts to the original three-dimensional model and addition of parts to the copied three-dimensional model, to integrate the original three-dimensional model and the copied three-dimensional model into the same three-dimensional model, wherein:
a flag is added to identification IDs of the parts to be added to the copied three-dimensional model after copying the original three-dimensional model; and
when the processing of adding the parts to the original three-dimensional model and the processing of adding the parts to the copied three-dimensional model are performed and there is a difference in the contents of the parts having the same identification ID, the identification ID is changed according to the flag so that the identification ID of the part added to the original three-dimensional model and the identification ID of the part added to the copied three-dimensional model become different, and the original three-dimensional model and the copied three-dimensional model are integrated into the same three-dimensional model.

2. A three-dimensional CAD system which allots identification IDs to respective component parts, references to already overlaid parts to overlay the part so to configure an original three-dimensional model, copies the original three-dimensional model, and concurrently performs addition and correction of parts to the original three-dimensional model and addition of parts to the copied three-dimensional model, to integrate the original three-dimensional model and the copied three-dimensional model into the same three-dimensional model, wherein:
a first flag is given to the identification ID of the part configuring the original three-dimensional model at a time when the original three-dimensional model is copied;
a second flag is given to the identification ID of the part to be added to the copied three-dimensional model after the original three-dimensional model is copied; and
when the processing of adding and correcting the parts to the original three-dimensional model and the processing of adding the parts to the copied three-dimensional model are performed and there is a difference in the contents of the parts having the same identification ID, the part having the identification ID which is given the first flag is judged as a part corrected in the original three-dimensional model, the part having the identification ID which is given the second flag is judged as a part added to the copied three-dimensional model, the identification ID is changed so that the identification ID of the part added to the original three-dimensional model and the identification ID of the part added to the copied three-dimensional model become different, and the original three-dimensional model and the copied three-dimensional model are integrated into the same three-dimensional model.

3. A three-dimensional CAD system which allots identification IDs to respective component parts, references to already overlaid parts to overlay the part so to configure an original three-dimensional model, copies the original three-dimensional model, and concurrently performs addition and correction of parts to the original three-dimensional model by a first design department and addition and correction of parts to the three-dimensional model copied by a second design department, to integrate the original three-dimensional model of the first design department and the three-dimensional model copied by the second design department into the same three-dimensional model, wherein:
a first flag is given to the identification ID of the part configuring the original three-dimensional model at a time when the original three-dimensional model is copied for a first time;
a second flag is given to the identification ID of the part to be added to the copied three-dimensional model after the original three-dimensional model is copied for the first time;
a third flag is given to the identification ID of the part configuring the original three-dimensional model at a time when the original three-dimensional model is copied for a second time;
the identification ID of the part configuring the three-dimensional model copied for the first time and the identification ID of the part configuring the three-dimensional model copied for the second time are compared and the contents are compared for differences;
it is judged that a part is not added or corrected when the first and third flags are given but the second flag is not given and there is not a content difference;
it is judged that the part is corrected by the first design department when the first and third flags are given but the second flag is not given and there is a content difference;
it is judged that the part is added by the first design department when the third flag is given but the first and second flags are not given;
it is judged that the part is added by the second design department when the second flag is given but the first and third flags are not given;
when the second and third flags are given but the first flag is not given and there is a content difference, it is judged that the parts are added by the first and second design departments, and the identification ID is changed so that the identification ID of the part becomes different; and
the original three-dimensional model of the first design department and the three-dimensional model copied by the second design department are integrated into the same three-dimensional model.

4. The three-dimensional CAD system according to claim 1, 2 or 3, wherein:
the first part has a relationship to refer to the second part and, when only the first part of these parts is integrated;
data that the identification ID of the second part is unnecessary is stored, and the identification IDs of both of the parts are changed;
the identification ID of the second part is deleted according to the stored data; and
the reference relationship is changed so that the first part refers to another part.
